(19) United States
(12) Patent Application Publication
(10) Pub. No.: US 2023/0242640 A1
(43) Pub. Date: Aug. 3, 2023

(54) ANTI-SIRPg Compounds

(71) Applicant: OSE IMMUNOTHERAPEUTICS, NANTES (FR)

(72) Inventors: NICOLAS POIRIER, Grandchamps des Fontaines (FR); CAROLINE MARY, SAINTE-PAZANNE (FR); Virginie THEPENIER, SAINTE-PAZANNE (FR); BERNARD VANHOVE, REZE (FR)

(21) Appl. No.: 17/270,028

(22) PCT Filed: Aug. 2, 2019
(86) PCT No.: PCT/EP2019/072527

§ 371 Date: Feb. 21, 2021

(30) Foreign Application Priority Data
Aug. 22, 2018 (EP) 18306131.6

(51) Int. Cl. C07K 16/28; A61K 39/395; A61K 45/06; A61P 37/06

(52) U.S. Cl. CPC C07K 16/2803; A61K 39/3955; A61K 45/06; A61P 37/06; C07K 2317/33; C07K 2317/73; C07K 2317/76; C07K 2317/92

(57) ABSTRACT

The invention pertains to the field of immunotherapy. The present invention provides new anti-SIRPg compounds, which are suitable for the treatment and/or the prevention of autoimmune disorders or diseases.

### FIELD OF THE INVENTION

The invention pertains to the field of immunotherapy. The present invention provides new specific anti-SIRPg compounds, in particular antibodies, which are able to antagonize the binding of SIRPg to CD47, without decreasing the interaction between the other members of the SIRP family and their target(s), e.g. the interaction between SIRPa and CD47.

The Sequence Listing in the ASCII text file named B13476WO_ST_25_A.txt created on Sep. 9, 2021, which is 19.1 KB, is incorporated herein by reference.

### BACKGROUND OF THE INVENTION

Signal-regulatory proteins (SIRPs) constitute a family of transmembrane glycoproteins widely expressed in the immune and central nervous system and that transduce different signals.

The prototypical member of the SIRP family is SIRP-alpha (also designated as SIRPa, SIRPα, CD172a or SHPS-1). The gene coding for human SIRPa is a polymorphic gene and several variants were described in human population. The most common protein variants are SIRPa v1 (Accession number NP_542970 and P78324) and SIRPa v2 (Accession number CAA71403). SIRPa is expressed on monocytes, most subpopulations of tissue macrophages, granulocytes, subsets of dendritic cells in lymphoid tissues, some bone marrow progenitor cells, and to varying levels on neurons, with a notably high expression in synapse-rich areas of the brain, such as the granular layer of the cerebellum and the hippocampus. SIRPa is an inhibitory receptor that binds CD47 and modulates macrophage and dendritic cell function, as well as signaling pathways induced by growth factors and cell adhesion.

Another member of the SIRP family, SIRP-beta (also designated SIRPb, SIRPβ, CD172b or SIRP beta - 1 - Accession number NM_001083910 or Accession Q5TFQ8), was also identified. Unlike other members of the SIRP family, SIRPb does not seem to bind to CD47, and its ligand is not known yet. Nonetheless, SIRPb seems associated with the killer cell immunoglobulin-like receptor family and may act as an activating signal transduction element. It has also been reported that SIRPb participate in the recruitment of tyrosine kinase SYK.

The third member of the SIRP family, SIRP-gamma (also designated as SIRPg, SIRPγ, CD172g or SIRP beta 2 - Accession number NM_018556 or Accession number Q9P1W8) was later identified. SIRPg is variably expressed in many human tissues, but in particular at the surface of T cells. Authors conclude that the SIRPg-CD47 interaction mediates cell-cell adhesion, enhances superantigen-dependent T-cell-mediated proliferation and co-stimulates T-cell activation (Piccio et al., Blood, 105:6, 2005), and SIRPg is involved in the activation of NK cells. However, it appears that anti-SIRPg antibody can in some conditions partially inhibit the proliferation of T cells. SIRPg-CD47 interaction also supports Antigen Presenting Cells - T cells contact, enhancing antigen presentation, the consequent T cell proliferation, and cytokine secretion.

In this context, the Inventors provide new anti-SIRPg compounds, which recognize and bind specifically to human SIRPg, in particular which prevent or inhibit the binding of SIRPg to CD47, and which in particular do not bind to the other members of the SIRP family, namely human SIRPa and human SIRPb. Indeed, there is a need for a compound which is able to selectively bind human SIRPg, with no or little recognition of the other members of the SIRP family, namely human SIRPa and SIRPb and which is an antagonist of the binding of human SIRPg to human CD47.Such compounds are inhibitors of the interaction between human SIRPg and human CD47, thereby inhibiting or preventing the binding of human SIRPg to human CD47, in particular without impairing the signalization triggered or modulated by the other members of the SIRP family, in particular the signalization triggered by SIRPa. Such specific anti-SIRPg compounds of the invention may have a stronger effect on the inhibition of T cells proliferation than cross-reacting antibodies that recognize both SIRPg and SIRPa, or anti-SIRPg antibodies which does not bind SIRPg on the same localization. Such specific anti-SIRPg compounds of the invention may also exhibit less side effects than cross-reacting anti-SIRP antibodies (i.e. antibodies which recognize several members of the SIRP family).

Such compounds are particularly suitable for their uses in the prevention and/or the treatment of several diseases, in particular diseases wherein T cells are involved (in which T cells have a deleterious effect), in particular for modulating T cells proliferation and/or activation and/or migration and/or tissues infiltration by T cells, in particular wherein acting on the proliferation and/or the activation and/or the migration of T cells and/or tissues infiltration by T cells may improve the outcome of the disease.

### SUMMARY OF THE INVENTION

In a first aspect, the invention relates to a specific anti-SIRPg compound selected from the group consisting of an antibody, an antigen-binding fragment thereof and an antigen binding antibody mimetic which specifically binds to an epitope of human SIRPg consisting of or localized within the polypeptide of sequence SEQ ID No: 1, which is an antagonist of the binding of human SIRPg to human CD47, and which is suitable for use in the prevention and/or treatment of a disease or a disorder, in particular a human disease or a human disorder, in which T cells have a deleterious effect. In a particular embodiment of the invention, the anti-SIRPg compound is useful in the treatment and/or the prevention of a disease or a disorder, in particular a human disease or a human disorder, in which the proliferation and/or the activation and/or the migration of T cells and/or tissues infiltration by T cells has(have) a deleterious effect. In particular embodiments of the invention, the term “localized” may be susbstituted by any one of the terms “present” or “comprised” “or “included”. In a more particular aspect, the invention relates to an anti-SIRPg compound selected from the group consisting of an antibody, an antigen-binding fragment thereof, or an antigen binding antibody mimetic which specifically binds to an epitope sequence of human SIRPg consisting of at least 10 consecutive amino acid residues which share at least 80% identity with SEQ ID No: 1, with the proviso that the amino acid residues “E” and “N” localized respectively on positions 9 and 10 of SEQ ID No: 1, and/or the amino acid residue “P” localized on position 17 of SEQ ID No: 1, and/or the amino acid residues “M”, “A”, “L” and “G” localized respectively on positions 21 to 24 of SEQ ID No: 1, and/or the amino acid residues of SEQ ID No. 45, are present, and in particular which is an antagonist of the binding of human SIRPg to human CD47.

In another particular aspect, the invention relates to a specific anti-SIRPg compound selected from the group consisting of an antibody, an antigen-binding fragment thereof and an antigen-binding antibody mimetic which specifically binds to a polypeptide, for example a linear polypeptide, consisting of amino acid residues of SEQ ID No: 1; or consisting of amino acid residues of SEQ ID No. 45, and in particular which is an antagonist of the binding of human SIRPg to human CD47.

Anti-SIRPg compounds which specifically bind to the polypeptide of SEQ ID No: 1 or SEQ ID No. 45 as defined here above, and/or which specifically bind to an epitope sequence as defined here above, are able to antagonize the binding of human SIRPg to human CD47, while they show a specific binding for SIRPg, and do not bind to the other members of the SIRP family, namely SIRPa and SIRPb. In other words, such a specific anti-SIRPg compound has the capability to prevent or inhibit the binding of human SIRPg to human CD47 and/or prevents the activation or inhibits the signaling pathway induced when SIRPg binds to CD47 in absence of a compound according to the invention, without impairing the signalization triggered or modulated by the other members of the SIRP family, in particular the signalization triggered by SIRPa. The inventors found that anti-SIRPg compounds, like but not limited to antibodies, antigen-binding fragments thereof, antigen-binding antibody mimetics thereof, which specifically bind to a peptide consisting of or localized within the amino acid residues of SEQ ID No. 1 or SEQ ID No. 45 have the ability to antagonize the binding of SIRPg to its target CD47 (or inhibit the binding of SIRPg to CD47), and have a binding specificity for SIRPg, more particularly for human SIRPg, and do not specifically bind to SIRPa and SIRPb, in particular to human SIRPa and human SIRPb. Such specific anti-SIRPg compounds of the invention may have a stronger effect on the inhibition of T cells proliferation than cross-reacting antibodies that recognize both SIRPg and SIRPa, or anti-SIRPg antibodies which does not bind SIRPg on the same localization. Such specific anti-SIRPg compounds of the invention may also exhibit less side effects than cross-reacting anti-SIRP antibodies (i.e. antibodies which recognize several members of the SIRP family).

In another aspect, the invention relates to a specific anti-SIRPg compound as described above, for its use in the prevention and/or the treatment of a disease or a disorder in which T cells have deleterious effects, in particular in which the proliferation and/or the activation and/or the migration of T cells and/or tissues infiltration by T cells has(have) a deleterious effect, wherein said anti-SIRPg compound is an antagonist of the interaction between human SIRPg and human CD47; in other words, wherein said compound inhibits and/or prevents the binding of human CD47 to human SIRPg; and wherein said compound does not bind to any other member of the SIRP family, namely SIRPa SIRPb, therefore does not impair the signalization triggered or modulated by the other members of the SIRP family, in particular the signalization triggered by SIRPa (notably via the interaction of SIRPa with CD47).

In another aspect, the invention relates to a specific anti-SIRPg compound as described above, for its use in the decrease or inhibition of T cells proliferation and/or activation and/or migration and/or tissues infiltration as compared with a negative control. In another aspect, the invention relates to a specific anti-SIRPg compound as described above, for its use in the decrease or inhibition of T cells proliferation as compared with a negative control. In another aspect, the invention relates to an anti-SIRPg compound as described above for its use in the decrease or inhibition of T cells activation as compared with a negative control. In another aspect, the invention relates to a specific anti-SIRPg compound as described above, for its use in the decrease or inhibition of T cells migration as compared with a negative control. In another aspect, the invention relates to a specific anti-SIRPg compound as described above, for its use in the decrease or inhibition of T cells tissues infiltration as compared with a negative control. In another aspect, the invention relates to an anti-SIRPg compound as described above for its use in the decrase or inhibition of T cells activation and the decrease or inhibition of T cells proliferation as compared with a negative control. In another aspect, the invention relates to an anti-SIRPg compound as described above for its use in the decrease or inhibition of T cells migration and the decrease or inhibition of T cells tissues infiltration as compared with a negative control. In another aspect, the invention relates to an anti-SIRPg compound as described above for its use in the decrease or inhibition of T cells proliferation and activation and migration and tissues infiltration by T cells as compared with a negative control. In particular, the invention relates to an anti-SIRPg compound as described above for its use in the reduction of the engraftment of T cells, leukocytes and/or NK-cells after a transplantation or during an inflammatory disease. In particular, the invention relates to an anti-SIRPg compound as described above that allows an enhanced survival of transplanted animals, in particular a human, by inhibiting the proliferation and/or the activation and/or the migration and/or the tissues inflitration of T cells within the transplanted animal.

In another aspect, the invention relates to the use of a specific anti-SIRPg compound as described above for the treatment of an immune system disorder, or an inflammatory disease, in particular graft-versus-host disease (GVHD), in particular acute and/or chronic GVHD, wherein activation and/or proliferation of T cells has a deleterious effect. Allogenic transplantation involves the transfer of cells or an organ from a donor to a genetically different recipient. The main clinical complication after such a transplantation is the development of GVHD, an immunological disorder mediated by donor T cells. Donor T cells may be toxic to the recipient and have the potential to attack and damage multiple organs and tissues of the allo-transplanted recipient, resulting in a high risk for morbidity and mortality. The use of anti-SIRPg compound as described above could reduce the proliferation and/or activation of T cells within a GVHD model. The proliferation of T-cells may be determined by various methods. For example, the proliferation of T-cells can be measured by incorporation of H3-thymidine in presence and in absence of an anti-SIRPg compound according to the invention. In particular, it is considered that an anti-SIRPg compound as described above decreases or inhibits the proliferation of T-cells when the proliferation of T-cells is over 20%, more preferably over 40%, more preferably over 50% and most preferably over 70% as compared with a negative control. A negative control may consist in the same assay, wherein a similar compound (e.g. an antibody, an antigen-binding fragment thereof, an antigen-binding antibody mimetic) which does not interfere with the CD47/SIRPg binding nor with the signaling pathway induced when SIRPg binds to CD47, is used in a similar amount in the assay instead of the anti-SIRPg compound. The anti-SIRPg compound as described above may be used within the context of an immune-suppressive therapy, in particular to prevent or treat clinical conditions related to transplantation associated GVHD or transfusion GVHD. The anti-SIRPg compound as described above may also be used for a prophylactic treatment against GVHD. In another aspect, the invention relates to the use of an anti-SIRPg compound as described above for delaying inflammatory disease progression. In another aspect, the invention relates to the use of an anti-SIRPg compound as described above for delaying unwanted lymphoproliferative events such as T cell lymphoma of post-transplant lymphoproliferative disease.

### DETAILED DESCRIPTION OF THE INVENTION

As used herein, the term “antibody” refers to polyclonal antibodies, monoclonal antibodies or recombinant antibodies.

As used herein, a “monoclonal antibody” is intended to refer to a preparation of antibody molecules, antibodies which share a common heavy chain and common light chain amino acid sequence, in contrast with “polyclonal” antibody preparations which contain a mixture of antibodies of different amino acid sequence. Monoclonal antibodies can be generated by several known technologies like phage, bacteria, yeast or ribosomal display, as well as by classical methods exemplified by hybridoma-derived antibodies. Thus, the term “monoclonal” is used to refer to all antibodies derived from one nucleic acid clone.

The antibodies of the present invention include recombinant antibodies. As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human. In an embodiment, the antibodies of the invention are chimeric antibodies.

In an embodiment, the antibodies of the invention are humanized antibodies.

As used herein, a “humanized antibody” refers to an antibody in which CDR sequences derived from the germline of another mammalian species, such as a mouse, have been grafted onto human framework sequences.

As used herein, an “antigen-binding fragment of an antibody” means a part of an antibody, i.e. a molecule corresponding to a portion of the structure of the antibody of the invention, that exhibits antigen-binding capacity for SIRPg, possibly in its native form; such fragment especially exhibits the same or substantially the same antigen-binding specificity for SIRPg compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies.

Antigen-binding fragments of antibodies are fragments which comprise their hypervariable domains designated CDRs (Complementary Determining Regions) or part(s) thereof encompassing the recognition site for the antigen, i.e. the extracellular domain of SIRPg, in particular the epitope sequence localized within SEQ ID No: 1 and defined herein, thereby defining antigen recognition specificity.

Each Light and Heavy chain variable domains (respectively VL and VH) of a four-chain immunoglobulin has three CDRs, designated VL-CDR1 (or LCDR1), VL-CDR2 (or LCDR2), VL-CDR3 (or LCDR3) and VH-CDR1 (or HCDR1), VH-CDR2 (or HCDR2), VH-CDR3 (or HCDR3), respectively.

The skilled person is able to determine the location of the various regions/domains of antibodies by reference to the standard definitions in this respect set forth, including a reference numbering system, a reference to the numbering system of KABAT or by application of the IMGT “collier de perle” algorithm. In this respect, for the definition of the sequences of the invention, it is noted that the delimitation of the regions/domains may vary from one reference system to another. Accordingly, the regions/domains as defined in the present invention encompass sequences showing variations in length or localization of the concerned sequences within the full-length sequence of the variable domains of the antibodies, of approximately +/- 10%.

Based on the structure of four-chain immunoglobulins, antigen-binding fragments can thus be defined by comparison with sequences of antibodies in the available databases and prior art, and especially by comparison of the location of the functional domains in these sequences, noting that the positions of the framework and constant domains are well defined for various classes of antibodies, especially for IgGs, in particular for mammalian IgGs. Such comparison also involves data relating to 3-dimensional structures of antibodies.

For illustration purpose of specific embodiments of the invention, antigen binding fragments of an antibody that contain the variable domains comprising the CDRs of said antibody encompass Fv, dsFv, scFv, Fab, Fab′, F(ab′)2. Fv fragments consist of the VL and VH domains of an antibody associated together by hydrophobic interactions; in dsFv fragments, the VH:VL heterodimer is stabilized by a disulphide bond; in scFv fragments, the VL and VH domains are connected to one another via a flexible peptide linker thus forming a single-chain protein. Fab fragments are monomeric fragments obtainable by papain digestion of an antibody; they comprise the entire L chain, and a VH-CH1 fragment of the H chain, bound together through a disulfide bond. The F(ab′)2 fragment can be produced by pepsin digestion of an antibody below the hinge disulfide; it comprises two Fab′ fragments, and additionally a portion of the hinge region of the immunoglobulin molecule. The Fab′ fragments are obtainable from F(ab′)2 fragments by cutting a disulfide bond in the hinge region. F(ab′)2 fragments are divalent, i.e. they comprise two antigen binding sites, like the native immunoglobulin molecule; on the other hand, Fv (a VH:VL dimmer constituting the variable part of Fab), dsFv, scFv, Fab, and Fab′ fragments are monovalent, i.e. they comprise a single antigen-binding site. These basic antigen-binding fragments of the invention can be combined together to obtain multivalent antigen-binding fragments, such as diabodies, tribodies or tetrabodies. These multivalent antigen-binding fragments are also part of the present invention.

As used herein, the term “bispecific” antibodies refers to antibodies that recognize two different antigens by virtue of possessing at least one region (e.g. derived from a variable region of a first antibody) that is specific for a first antigen, and at least a second region (e.g. derived from a variable region of a second antibody) that is specific for a second antigen. A bispecific antibody specifically binds to two target antigens and is thus one type of multispecific antibody. Multispecific antibodies, which recognize two or more different antigens, can be produced by recombinant DNA methods or include, but are not limited to, antibodies produced chemically by any convenient method. Bispecific antibodies include all antibodies or conjugates of antibodies, or polymeric forms of antibodies which are capable of recognizing two different antigens. Bispecific antibodies include antibodies that have been reduced and reformed so as to retain their bivalent characteristics and to antibodies that have been chemically coupled so that they can have several antigen recognition sites for each antigen such as BiME (Bispecific Macrophage Enhancing antibodies), BiTE (bispecific T cell engager), DART (Dual affinity retargeting); DNL (dock-and-lock), DVD-lg (dual variable domain immunoglobulins).

Accordingly, bispecific antibodies of the invention are directed against SIRPg and a second target, which in particular is neither SIRPa nor SIRPb.

In an embodiment, the anti-SIRPg compound of the invention is bispecific, in particular is a bispecific antibody.

Several researches to develop therapeutic antibodies had led to engineer the Fc regions to optimize antibody properties allowing the generation of molecules that are better suited to the pharmacology activity required of them. The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Several mutations located at the interface between the CH2 and CH3 domains, such as T250Q/M428L and M252Y/S254T/T256E + H433K/N434F, have been shown to increase the binding affinity to FcRn and the half-life of lgG1 in vivo. However, there is not always a direct relationship between increased FcRn binding and improved half-life. One approach to improve the efficacy of a therapeutic antibody is to increase its serum persistence, thereby allowing higher circulating levels, less frequent administration and reduced doses. Engineering Fc regions may be desired to either reduce or increase the effector function of the antibody. For antibodies that target cell-surface molecules, especially those on immune cells, abrogating effector functions is required. Conversely, for antibodies intended for oncology use, increasing effector functions may improve the therapeutic activity. The four human IgG isotypes bind the activating Fcy receptors (FcyRI, FcyRlla, FcyRllla), the inhibitory FcyRllb receptor, and the first component of complement (C1q) with different affinities, yielding very different effector functions. Binding of IgG to the FcyRs or C1q depends on residues located in the hinge region and the CH2 domain. Two regions of the CH2 domain are critical for FcyRs and C1q binding, and have unique sequences in lgG2 and lgG4.

As used herein, a “modified antibody” corresponds to a molecule comprising an antibody or an antigen-binding fragment thereof, wherein said monoclonal antibody or functional fragment thereof is associated with a functionally different molecule. A modified antibody of the invention may be either a fusion chimeric protein or a conjugate resulting from any suitable form of attachment including covalent attachment, grafting, chemical bonding with a chemical or biological group or with a molecule, such as a PEG polymer or another protective group or molecule suitable for protection against proteases cleavage in vivo, for improvement of stability and/or half-life of the antibody or functional fragment. With similar techniques, especially by chemical coupling or grafting, a modified antibody can be prepared with a biologically active molecule, said active molecule being for example chosen among toxins, in particular Pseudomonas exotoxin A, the A-chain of plant toxin ricin or saporin toxin, especially a therapeutic active ingredient, a vector (including especially a protein vector) suitable for targeting the antibody or functional fragment to specific cells or tissues of the human body, or it may be associated with a label or with a linker, especially when fragments of the antibody are used. PEGylation of the antibody or functional fragments thereof is a particular interesting embodiment as it improves the delivery conditions of the active substance to the host, especially for a therapeutic application. PEGylation can be site specific to prevent interference with the recognition sites of the antibodies or functional fragments, and can be performed with high molecular weight PEG. PEGylation can be achieved through free cysteine residues present in the sequence of the antibody or functional fragment or through added free Cysteine residues in the amino sequence of the antibody or functional fragment.

In an embodiment, the anti-SIRPg compound of the invention is modified, in particular is a modified antibody.

A compound according to the invention may also be a macromolecule. Macromolecules usually comprise antibodies and/or fragments thereof but also comprise artificial proteins with the capacity to bind antigens mimicking that of antibodies, also termed herein antigen-binding antibody mimetic.

Antigen-binding antibody mimetics are organic compounds that specifically bind antigens, but that are not structurally related to antibodies. They are usually artificial peptides or small proteins with a molar mass of about 3 to 20 kDa. Nucleic acids and small molecules are sometimes considered antibody mimetics as well, but not artificial antibodies, antibody fragments and fusion proteins composed from these. Common advantages over antibodies are better solubility, tissue penetration, stability towards heat and enzymes, and comparatively low production costs. Antibody mimetics are being developed as therapeutic and diagnostic agents. Antigen-binding antibody mimetics may also be selected among the group comprising affibodies, affilins, affimers, affitins, DARPins, and Monobodies.

An antigen-binding antibody mimetic is more preferentially selected from the group comprising affitins and anticalins. Affitins are artificial proteins with the ability to selectively bind antigens. They are structurally derived from the DNA binding protein Sac7d, found in Sulfolobus acidocaldarius, a microorganism belonging to the archaeal domain. By randomizing the amino acids on the binding surface of Sac7d, e.g. by generating variants corresponding to random substitutions of 11 residues of the binding interface of Sac7d, an affitin library may be generated and subjecting the resulting protein library to rounds of ribosome display, the affinity can be directed towards various targets, such as peptides, proteins, viruses and bacteria. Affitins are antibody mimetics and are being developed as tools in biotechnology. They have also been used as specific inhibitors for various enzymes (Krehenbrink et al., J. mol. Biol., 383:5, 2008). The skilled person may readily develop affitins with the required binding properties using methods know in the art, in particular as disclosed in patent application WO2008068637 and the above-cited publication, in particular the generation of phage display and/or ribosome display libraries and their screening using an antigen as disclosed herein. Anticalins are artificial proteins that are able to bind to antigens, either to proteins or to small molecules. They are antibody mimetic derived from human lipocalins which are a family of naturally binding proteins. Anticalins are about eight times smaller with a size of about 180 amino acids and a mass of about 20 kDa (Skerra, Febs J., 275:11, 2008). Anticalin phage display libraries have been generated which allow for the screening and selection, in particular of anticalins with specific binding properties. The skilled person may readily develop anticalins with the required binding properties using methods know in the art, in particular as disclosed in EP patent EP1270725 B1, U.S. Pat. US8536307 B2, Schlehuber and Skerra, Biophys. Chem., 96:2-3, 2002 and the above-cited publication, in particular the generation of phage display and/or ribosome display libraries and their screening using an antigen as disclosed herein. Anticalins and affitins may both be produced in a number of expression system comprising bacterial expression systems. Thus, the invention includes affitins, anticalins and other similar antibody mimetics with the features of the antibodies described herein, in particular with regard to binding to SIRPg, in particular the binding to a polypeptide or the epitope sequence as defined herein, to the inhibition of the binding of CD47 to SIRPg, to the absence of binding to SIRPa and/or SIRPb, all of which are contemplated as compounds of the invention.

All the embodiments disclosed herein for antibodies or fragments thereof are transposed mutatis mutandis to the compounds of the invention, in particular to antigen-binding antibody mimetic, humanized antibodies, modified antibodies and chimeric antibodies.

As used herein, the term “epitope” means the part of an antigen to which the antibody binds. The epitopes of protein antigens can be divided into two categories, conformational epitope and linear epitope. A conformational epitope corresponds to discontinuous sections of the antigen’s amino acid sequence. A linear epitope corresponds to a continuous sequence of amino acids from the antigen.

In the invention, the polypeptide corresponding to a sequence presents within SIRPg and that is bound by the anti-SIRPg compounds comprises an epitope specifically recognized by these compounds.

As used herein, the term “SIRPg” relates to a SIRPg from a mammal species, preferably a human SIRPg.

A reference sequence of the human SIRPg protein corresponds to the sequence associated to the Accession number Q9P1W8 or NM 018556.

In the following description of the invention, the term anti-SIRPg compound means either an antibody (e.g. a humanized antibody, a chimeric antibody, a modified antibody), an antigen-binding fragment, an antigen-binding antibody mimetic,, or a macromolecule as defined here above. When the term anti-SIRPg antibody is used, the same compounds are encompassed by this term, except when specified in relation to a particular embodiment of the invention.

A “specific anti-SIRPg compound” is a compound that exhibits specific binding for SIRPg and which does not exhibit specific binding for SIRPa, in particular which does not exhibit specific binding for SIRPa and SIRPb, binding being in each case detectable by methods known in the art like but not limited to Biacore analysis, Blitz analysis, ELISA assay or Scatchard plot. A specific “anti-SIRPg compound” may also be defined as an antibody that exhibits low binding affinity for SIRPg but that nevertheless antagonize the binding of SIRPg to CD47 (e.g. blocks the interaction between CD47 and SIRPg), while it does not exhibit binding to SIRPa, in particular it does not exhibit specific binding to SIRPa and SIRPb, and does not prevent the interaction between CD47 and SIRPa . By “block the interaction” it should be understood that the compound has an antagonist effect on the CD47/SIRPg interaction and, in a particular embodiment prevents or inhibits the binding of human SIRPg to human CD47 and/or prevents the activation or inhibits the signaling pathway induced when SIRPg binds to CD47 in absence of a compound according to the invention. In a particular embodiment, a specific binding may correspond to EC50 for SIRPg as recited in paragraph 44 or 45. Moreover, such specific binding may also be defined in relation with EC50 for SIRPa, as recited in paragraph 42, in particular EC50 for SIRPa and SIRPb, as recited in paragraph 42 and 43. In a particular embodiment, by specific anti-SIPRg compound, it should be understood that such a compound has at least one feature listed in the following paragraphs (from to [49]).

In a particular embodiment, a specific anti-SIRPg compound according to the invention has an EC50 value for SIRPa over 5 ug/ml, more particularly over 6 ug/ml, more particularly over 7 µg/ml, more particularly over 8 µg/ml, more particularly over 9 ug/ml, and most particularly over 10 ug/ml. The EC50 may be determined according to methods known in the art, or by the method disclosed in the examples of the present invention.

In a particular embodiment, a specific anti-SIRPg compound according to the invention has an EC50 value for SIRPb over 5 ug/ml, more particularly over 6 ug/ml, more particularly over 7 µg/ml, more particularly over 8 µg/ml, more particularly over 9 ug/ml, and most particularly over 10 µg/ml. The EC50 may be determined according to methods known in the art, or by the method disclosed in the examples of the present invention.

In another particular embodiment, a specific anti-SIRPg compound according to the invention has an EC50 value for SIRPg comprised between 100 ng/ml and 1000 ng/ml, more particularly between between 100 ng/ml and 800 ng/ml. The EC50 may be determined according to methods known in the art, or by the method disclosed in the examples of the present invention.

In another particular embodiment, a specific anti-SIRPg compound according to the invention has an EC50 value for SIRPg lower than 1000 ng/ml, more particularly lower than 800 ng/ml. The EC50 may be determined according to methods known in the art, or by the method disclosed in the examples of the present invention.

In another particular embodiment, a specific anti-SIRPg compound according to the invention has an IC50 value for the binding between SIRPg and CD47 comprised between 20 ng/ml and 2000 ng/ml, more particularly between 20 ng/ml and 1000 ng/ml, more particularly between 20 ng/ml and 400 ng/ml. The IC50 value of the binding between SIRPg and CD47 may be assessed according to methods known in the art, or by the method disclosed in the examples of the invention.

In another particular embodiment, a specific anti-SIRPg compound according to the invention has an IC50 value for the binding between SIRPg and CD47 lower than 2000 ng/ml, more particularly lower than 1000 ng/ml, more particularly lower than 400 ng/ml. The IC50 value of the binding between SIRPg and CD47 may be assessed according to methods known in the art, or by the method disclosed in the examples of the invention.

In another particular embodiment, a specific anti-SIRPg compound according to the invention has an EC50 value for SIRPg lower than 1000 ng/ml, more particularly lower than 800 ng/ml, and an IC50 value for the binding between CD47 and SIRPg lower than 2000 ng/ml, more particularly lower than 1000 ng/ml, more particularly lower than 400 ng/ml.

In another particular embodiment, a specific anti-SIRPg compound according to the invention has an EC50 value for SIRPg lower than 1000 ng/ml, more particularly lower than 800 ng/ml, and an EC50 value for SIRPa over 5 ug/ml, more particularly over 6 ug/ml, more particularly over 7 µg/ml, more particularly over 8 ug/ml, more particularly over 9 ug/ml, and most particularly over 10 µg/ml.

The term “IC50” and as used herein refers to the measure of the effectiveness of a compound (e.g., an anti-SIRPg compound) in inhibiting a biological or biochemical function (e.g., the function or activity of SIRPg) by 50%. For example, IC50 indicates how much of an anti-SIRPg compound is needed to inhibit the activity of SIRPg by half. That is, it is the half maximal (50%) inhibitory concentration (IC) of an anti-SIRPg compound (50% IC, or IC50). IC50 represents the concentration of a drug that is required for 50% inhibition in vitro. The IC50 can be determined by techniques known in the art, for example, by constructing a dose-response curve and examining the effect of different concentrations of the anti-SIRPg compound on reversing SIRPg activity. A method is for example disclosed in the examples of the present invention.

The term “EC50” and as used herein refers to the measure of the effectiveness of a compound (e.g., an anti-SIRPg compound) in eliciting a biological or biochemical function (e.g., the function or activity of SIRPg) by 50%. For example, EC50 indicates how much of an anti-SIRPg compound is needed to elicit the activity of SIRPg by half. That is, it is the half maximal (50%) effective concentration (EC) of an anti-SIRPg compound (50% EC, or EC50). EC50 represents the concentration of a drug that is required for 50% effectiveness in vitro. The EC50 can be determined by techniques known in the art, for example, by constructing a dose-response curve and examining the effect of different concentrations of the anti-SIRPg compound on SIRPg activity. A method is for example disclosed in the examples of the present invention.

T cell proliferation and T cell activation may be determined by various methods. For example, the proliferation of T cells can be measured by incorporation of H3-thymidine. In particular, it is considered that a compound does inhibit the proliferation of T-cells when the proliferation of T-cells is reduced by more than 20% compared to a negative control. The T cell activation may be assessed by analyzing the expression of CD25 and/or CD69, for example by flow cytometry, western blot, ELISA, and the like, and/or by assessing the secretion of IFNg and/or IL2, as compared to a control known for not activating T cells. T cells may be considered activated when expression of CD25 and/or CD69 is increased by at least 20% compared to a negative control. T cells may be considered activated when secretion of IFNg and/or IL2 is increased by at least 20% compared to a negative control. Tissues infiltration and migration of T cells may be assessed by counting the number of T cells within a particular localization within the body (e.g. a tissue, an organ, a fluid, a gland) over time in presence of an anti-SIRPg compound according to the invention as compared to a negative control. Tissues infiltration of T cells may be considered positive when the number of T cells increases over the time. Migration of T cells may be considered positive when the number of T cells vary over the time. T cells may be counted according to known method, like but not limited to the use of specific T cell markers.

In one aspect, the invention relates to a specific anti-SIRPg compound selected from the group consisting of an antibody (in particular a chimeric and a humanized antibody), an antigen-binding fragment thereof, an antigen binding antibody mimetic, which specifically binds to an epitope of human SIRPg consisting of or localized within the polypeptide consisting of sequence SEQ ID No: 1 (VKFRKGSPENVEFKSGPGTEMALGAKPSA), which is an antagonist of the binding of human SIRPg to human CD47, and which is suitable for use in the prevention and/or treatment of a disease or a disorder, in particular a human disease or a human disorder, in which T cells have a deleterious effect. In a particular embodiment, the invention relates to a specific anti-SIRPg compound selected from the group consisting of an antibody (in particular a chimeric and a humanized antibody), an antigen-binding fragment thereof, an antigen binding antibody mimetic, which specifically binds to an epitope of human SIRPg consisting of or localized within the polypeptide consisting of sequence SEQ ID No: 1 (VKFRKGSPENVEFKSGPGTEMALGAKPSA), which is an antagonist of the binding of human SIRPg to human CD47, and which is suitable for use in the prevention and/or treatment of a disease or a disorder, in particular a human disease or a human disorder, in which the proliferation and/or the activation and/or the migration of T cells and/or tissues infiltration by T cells has (have) a deleterious effect.

In a particular embodiment, the compound specifically binds to an epitope sequence of human SIRPg consisting of at least 10 consecutive amino acid residues which share at least 80% identity, more preferably at least 85%, more preferably at least 90%, more preferably at least 95%, with SEQ ID No: 1, or consisting of at least 10 consecutive amino acid residues of SEQ ID No: 10, with the proviso that the amino acid residues “E” and “N” localized respectively on positions 9 and 10 of SEQ ID No: 1, and/or the amino acid residue “P” localized in position 17 of SEQ ID No: 1; and/or the amino acid residues “M”, “A”, “L” and “G” localized respectively on positions 21 to 24 of SEQ ID No: 1 are present. In another particular embodiment, the compound specifically binds to an epitope sequence of human SIRPg comprising or consisting of the amino acid residues of SEQ ID No. 45.

In a particular embodiment, the invention relates to an anti-SIRPg compound selected from the group consisting of an antibody (in particular a chimeric and a humanized antibody), an antigen-binding fragment thereof, an antigen-binding antibody mimetic, which specifically binds to an epitope of human SIRPg consisting of SEQ ID No: 1, which is an antagonist of the binding of human SIRPg to human CD47, and which is suitable for use in the prevention and/or treatment of a disease or a disorder, in particular a human disease or a human disorder, in which T cells have a deleterious effect. In a particular embodiment, the invention relates to a specific anti-SIRPg compound selected from the group consisting of an antibody (in particular a chimeric and a humanized antibody), an antigen-binding fragment thereof, an antigen binding antibody mimetic, which specifically binds to an epitope of human SIRPg consisting of or localized within the polypeptide consisting of sequence SEQ ID No: 1 (VKFRKGSPENVEFKSGPGTEMALGAKPSA), which is an antagonist of the binding of human SIRPg to human CD47, and which is suitable for use in the prevention and/or treatment of a disease or a disorder, in particular a human disease or a human disorder, in which the proliferation and/or the activation and/or the migration of T cells and/or tissues infiltration by T cells has a deleterious effect.

A disease or a disorder in which T cells have deleterious effects may include accordingly any disease or disorder wherein the T cells proliferation and/or activation and/or migration and/or tissues infiltration by T cells has (have) deleterious effects.

In particular, a disease or a disorder in which T cells have deleterious effects is selected from the group consisting of:
an auto-immune disease, in particular rheumatoid arthritis, type I diabetes, lupus, psoriasis,
a chronic inflammatory disease, in particular Inflammatory bowel diseases including Crohn disease and Ulcerative colitis,
a chronic neuroinflammatory disease, in particular multiple sclerosis, encephalomyelitis,
an immune-metabolic disease, in particular type II diabetes,
a cardiovascular disease caused by a systemic inflammation, in particular atherosclerosis, and
a transplant dysfunction or rejection, in particular Graft-versus-host disease,
a lymphoproliferative disease, in particular T cell lymphoma or post-transplant lymphoproliferative disease.

Given that antagonist SIRPg compound can reduce or inhibit the proliferation of T cells, they can favor an immunosuppressive environment and be useful for the treatment of an autoimmune disorder or disease, a transplant dysfunction, or an inflammatory disease. Indeed, while the immune response is the host’s normal and protective response to an injury or a disease, it can also cause undesired damages when it turns against host’s cells.

In an embodiment, the invention relates to a specific anti-SIRPg antibody or antigen-binding fragment thereof or antigen binding antibody mimetic as defined above, for its use in the treatment and/or the prevention of a disease or disorder, including the delay in the development of a disease or disorder, selected from the group consisting of:
an auto-immune disease, in particular rheumatoid arthritis, type 1 diabetes, lupus, psoriasis,
a chronic inflammatory disease, in particular Inflammatory bowel diseases including Crohn disease and Ulcerative colitis,
a chronic neuroinflammatory disease, in particular multiple sclerosis, encephalomyelitis,
an immune-metabolic disease, in particular type II diabetes,
a cardiovascular disease caused by a systemic inflammation, in particular atherosclerosis,
a transplant dysfunction or rejection, in particular Graft-versus-Host disease, and
a lymphoproliferative disease, in particular T cell lymphoma or post-transplant lymphoproliferative disease.

In particular, an anti-SIRPg compound as defined above inhibits the SIRPg-CD47 signaling pathway in vitro and/or in vivo, and in particular inhibits or decreases T cells proliferation and/or activation and/or migration and/or tissues infiltration by T cells in vitro and/or in vivo, as compared with a negative control.

A binding between an anti-SIRPg compound and the epitope sequence as defined here above may be considered specific when the effective dose of the compound to reach 50% of the maximum signal (EC50) in a binding assay is lower than 1000 ng/ml, more particularly lower than 800 ng/ml, and still more particularly lower than 400 ng/ml. Such an ability may for example be assessed according to the methods illustrated in the examples of the present invention.

In a preferred embodiment, the epitope sequence of human SIRPg which is specifically recognized by an anti-SIRPg compound of the invention is constituted of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, or the 29 consecutive amino acid residues of SEQ ID No: 1. In a particular embodiment, irrespective of the length of the amino acid sequence, the epitope sequence shares at least 80% identity, more preferably at least 85%, more preferably at least 90%, more preferably at least 95%, with SEQ ID No: 1, and most preferably shares 100% identity with SEQ ID No: 1 . In a particular embodiment, the epitope sequence of human SIRPg which is specifically recognized by an anti-SIRPg compound of the invention comprises the amino acid residues “E” and “N” localized respectively on positions 9 and 10 of SEQ ID No: 1, the amino acid residue “P” localized on position 17 of SEQ ID No: 1, and the amino acid residues “M”, “A”, “L” and “G” localized respectively on positions 21 to 24 of SEQ ID No: 1, said epitope sequence comprising at least 16 amino acid residues, and have an identity with the other amino acid residues of SEQ ID No: 1 of at least 80%, more preferably at least 85%, more preferably at least 90%, more preferably at least 95%, with SEQ ID No: 1, and most preferably shares 100% identity with SEQ ID No: 1. In a particular embodiment, the epitope sequence of human SIRPg which is specifically recognized by an anti-SIRPg compound of the invention consists of the amino acid residues of SEQ ID No. 45.

According to a particular embodiment, the epitope sequence comprises or consists of the sequence of amino acid residues localized between positions 9 and 24 of SEQ ID No: 1.

In another particular embodiment of the invention, the epitope sequence comprises or consists of an amino acid sequence with an identity with SEQ ID No: 1 of at least 80%, more preferably at least 85%, more preferably at least 90%, most preferably at least 95%, with the proviso that the amino acid residues “E” and “N” localized respectively on positions 9 and 10 of SEQ ID No: 1, the amino acid residue “P” localized on position 17 of SEQ ID No: 1, and the amino acid residues “M”, “A”, “L” and “G” localized respectively on positions 21 to 24 of SEQ ID No: 1 are present, the epitope sequence having a length comprised between 16 and 29 amino acid residues.

In a particular embodiment, an anti-SIRPg compound according to the invention significantly inhibits, decreases, or competes with the binding of CD47 to SIRPg.

Therefore, the anti-SIRPg compound of the invention has the ability to inhibit the CD47 signaling pathway induced when SIRPg binds to CD47. The antagonist property, the disruption or the inhibition of the binding of SIRPg to CD47 may be assessed for example by competition ELISA assay, as described in the examples of the present invention. An anti-SIRPg compound may be considered as being an antagonist of the binding of SIRPg to CD47 when it allows over 60%, more preferably over 70%, more preferably over 80%, and most preferably over 90% of physical binding inhibition between CD47 and SIRPg at a concentration lower than 1000 ng/ml, more particularly lower than 800 ng/ml, and most particularly lower than 400 ng/ml. The same definition applies for an anti-SIRPg compound which is considered as preventing or inhibiting the binding of SIRPg to CD47. The antagonist effect can be determined using the methods illustrated in the examples of the present application.

In the invention, it can also be considered that an anti-SIRPg compound antagonizes the binding of CD47 to SIRPg if said compound induces an increase superior to 1 log, preferably superior to 2 log, more preferably superior to 3 log, most preferably superior to 4 log, of the KD value of CD47 in a SIRPg binding competitive assay, for example by Blitz or ELISA.

Such an anti-SIRPg compound has antagonist activity on the interaction between human SIRPg and human CD47. Such an ability may be assessed according to the method described here above, and/or by the methods described in the examples of the present invention. The antibodies of the invention also have the property of selectively binding to a sole member of the SIRP family, namely SIRPg, and does not specifically binds to the other members of the SIRP family, i.e. SIRPa and SIRPb, on the contrary to the antibodies of the prior art as illustrated in the examples of the present invention.The combination of properties recited herein (antagonist of the binding of SIRPg to CD47; specific binding to SIRPg ; no specific binding to SIRPa and SIRPb) are due to the binding of the anti-SIRPg compound of the invention to an epitope consisting of or localized within the amino acid residues of sequence SEQ ID No. 1 or SEQ ID No. 45..

The present description also discloses humanized anti-SIRPg antibodies. Indeed, an antibody or an antigen-binding fragment thereof, which is a humanized antibody can also be derived by substitution of amino acid residue(s) present in constant region(s) of variable chains (VH and/or VL), for human amino acid residue(s) having corresponding location in human antibodies according to standard definition and numbering, wherein the substitution level is from 1% to 20%, in particular from 1% to 18% of the residues in said framework regions. Said constant regions include those of framework regions (FRs) defined in four-chain antibodies identified in particular by reference to KABAT numbering.

Particular examples of modified antibodies according to the invention encompass chimeric antibodies, humanized antibodies and/or a de-immunized antibody. A deimmunized antibody is an antibody derived from a parental antibody wherein the primary structure is modified to lower its recognition by the immune system of the host, while the binding capability of the antibody is kept. Epitope sequences known to be recognized by MHC class II receptors (i.e. T cell epitopes) are sought within the primary structure of the antibody, and if such an epitope sequence is present, the sequence of the antibody is remodeled to remove the epitope sequence. Deimmunization consists in the identification of CD4+ T cell epitopes within the amino acid sequence of the antibody, and modification of the amino acid sequence when such an epitope is present. The identification step is performed with several algorithms. As an example, the following link (http://tools.iedb.org/main/tcell/) is a tool commonly used by the skilled person in the design of antibodies. Deimmunized antibodies, and methods for producing the same are for example illustrated in Durancel and Muller (mAbs 4:4, 445-457; July/August 2012; doi.org/10.4161/mabs.20776) or in Baker et al., (Methods in Molecular Biology ▪ February 2009 - DOI: 10.1007/978-1-59745-554-1_21).

Anti-SIRPg antibodies may be humanized according to known methods. As examples, the different combinations of CDRs of specific anti-SIRPg antibodies manufactured according to methods of the invention illustrated herein may be grafted on human heavy chain variable domain and/or light chain variable domain. The chimeric, humanized and/or de-immunized antibodies of the invention can belong to any class of immunoglobulins, like the non-modified antibodies. Preferably, they belong to a subclass of the IgG class such as IgG1, lgG2, lgG3 or lgG4.

Methods for preparing recombinant antibodies (or antigen-binding fragment thereof), or chimeric antibodies by combining the variable regions of an antibody with appropriate linkers, or with the constant regions of another antibody, are well known in the art.

In a particular embodiment of any aspect of the invention, the specific anti-SIRPg compound does not prevent or inhibit the binding of human SIRPa to human CD47, in particular the specific anti-SIRPg compound does not specifically bind to human SIRPa, and therefore does not block the interaction between human SIRPa and human CD47.

SIRPa is expressed on monocytes, most subpopulations of tissue macrophages, granulocytes, subsets of dendritic cells in lymphoid tissues, some bone marrow progenitor cells, and to varying levels on neurons, with a notably high expression in synapse-rich areas of the brain, such as the granular layer of the cerebellum and the hippocampus. The gene coding for human SIRPa is a polymorphic gene and several variants were described in human population. The most common protein variants are SIRPa v1 and v2 (accession numbers NP_542970 (P78324) and CAA71403). The polymorphisms in human SIRP lead to changes in surface-exposed amino acids, but this does not affect binding to CD47.

The SIRPa interaction with CD47 is largely described and provides a down regulatory signal that inhibits host cell phagocytosis. CD47 is widely expressed at lower levels by most healthy cells but it is also overexpressed in some cancer cells. Therefore, CD47 functions as a “don’t-eat-me” signal. Because CD47 serves as a “don’t-eat-me” signal and, as such, is an important determinant of host cell phagocytosis by macrophages, the potential contribution of CD47-SIRPa interaction in cancer cell clearance has been intensely investigated in recent years.

As used herein, the term “SIRPa” refers to a SIRPa protein from a mammal species, preferably a human SIRPa (e.g. accession numbers NP_542970 (P78324) and CAA71403).

According to this particular embodiment, a specific anti-SIRPg compound binds specifically to SIRPg, in particular human SIRPg, but not to SIRPa, in particular human SIRPa. In an embodiment, the anti-SIRPg compound has a KD value inferior to 10-8 M, preferably inferior to 10-9 M for SIRPa, particularly by Blitz Analysis or ELISA assay.

In a particular embodiment of any aspect of the invention, the specific anti-SIRPg compound does not specifically bind to human SIRPb.

As used herein, the term “SIRPb” refers to a SIRPb protein from a mammal species, preferably a human SIRPb (e.g. accession numbers NM_001083910 or Q5TFQ8).

In a particular embodiment of the invention, any specific anti-SIRPg compound described herein does not specifically bind to SIRPa and does not specifically bind to SIRPb.

In a particular embodiment of the invention, any anti-SIRPg compound described herein decreases or inhibits the proliferation of T cells due to its antagonist effect on the SIRPg-CD47 interaction. In a particular embodiment of the invention, such compound decreases or inhibits the proliferation of T cells as compared with a negative control, in particular the decrease or inhibition of the proliferation of T cells is over 20%, in particular over 40%, more particularly over 50%, and most particularly over 70%. A negative control may consist in the same assay, wherein a similar compound (e.g. an antibody, an antigen-binding fragment thereof, an antigen-binding antibody mimetic) which does not interfere with SIRPg, CD47, the CD47/SIRPg binding, and which does not interfere with the signaling pathway induced when SIRPg binds to CD47, is used in a similar amount in the assay instead of the anti-SIRPg compound.

Such a specific anti-SIRPg compound of the invention may have a stronger effect on the inhibition of T cells proliferation than cross-reacting antibodies that recognize both SIRPg and SIRPa, or anti-SIRPg antibodies which does not bind SIRPg on the same localization. Such a specific anti-SIRPg compound of the invention may also exhibit less side effects than cross-reacting anti-SIRP antibodies (i.e. antibodies which recognize several members of the SIRP family). Therefore, the present invention encompasses a specific anti-SIRPg compound which inhibits or decreases the binding of CD47 to SIRPg and/or which inhibits or decreases the proliferation of T-cells and which does not bind specifically to SIRPa and/or which does not inhibit the binding of CD47 to SIRPa, or a compound which inhibits the binding of CD47 to SIRPg and which inhibits or decreases the proliferation of T-cells, in particular CD4+ T cells, and which does not bind specifically to SIRPa and which does not inhibit the binding of CD47 to SIRPa, in particular which does not specifically bind to SIRPb. It should be noted that the inhibition or the decrease of the proliferation of T cells may be more important when such an anti-SIRPg compound is used instead of an anti-CD47 antibody. In a particular embodiment of the invention, such specific anti-SIRPg compound decreases or inhibits the proliferation of T cells as compared with a negative control, in particular the decrease or inhibition of the proliferation of T cells is over 20%, more preferentially over 40%, more preferentially over 50%, and most preferentially over 70%. Proliferation of T cells may be assessed by measure of the incorporation of H³-thymidine in presence and in absence of the anti-SIRPg compound.

In another aspect, the invention relates to the use of any anti-SIRPg compound described herein, for use in the treatment and/or the prevention of a disease, thereby in particular encompassing inhibiting, slowing the progression of, or reducing the symptoms associated with a disease or a disorder in which T cells has deleterious effects, in particular an autoimmune disease, a chronic inflammatory disease, a chronic neuroinflammatory disease, an immune-metabolic disease, a cardiovascular disease caused by a systemic inflammation or a transplant dysfunction. In a particular embodiment of the invention, a transplant dysfunction does not include graft rejection.

The anti-SIRPg compound can be administered alone or in combination with another therapeutic agent, e.g., a second human monoclonal antibody or antigen binding fragment thereof. In another example, the anti-SIRPg compound is administered together with another agent, for example, an immunotherapeutic agent, an immunosuppressive agent, an erythropoiesis-stimulating agent (ESA), a pro-apoptotic agent, antibiotic, probiotic, in combination with therapeutic cell compositions, and the like.

In an embodiment, the invention relates to an anti-SIRPg compound for its use as defined above, wherein the anti-SIRPg compound is combined with a second therapeutic agent.

The administration of the second agent can be simultaneous or not with the administration of the specific anti-SIRPg compound. Depending on the nature of the second agent, a co-administration can be prepared in the form of a combination drug, also known as a “combo”. A combo is a fixed-dose combination that includes two or more active pharmaceutical ingredients combined in a single dosage form, which is manufactured and distributed in fixed doses. But the dose regimen and/or the administration route can also differ.

In a preferred embodiment, this second therapeutic agent is selected from the group consisting of immunotherapeutic agents, immunosuppressive agents, pro-apoptotic agents, antibiotics and probiotics.

In a preferred embodiment, this second therapeutic agent is an immunosuppressive agent selected from the group consisting of Cyclosporine A, tacrolimus, mycophenolate mofetil, rapamycine, steroids, anti-TNF agents, anti-IL-23 agents.

The invention also relates to a combination product comprising:
at least one specific anti-SIRPg compound as defined above, and
at least one second therapeutic agent selected from the group consisting of immunotherapeutic agents, immunosuppressive agents, pro-apoptotic agents, antibiotics and probiotics,
for simultaneous, separate or sequential use as a medicament, in particular for the prevention and/or the treatment of a disease or a disorder in which the activation and/or proliferation of T cells has a deleterious effect.

The compound may be provided at an effective dose from about 1 ng/kg body weight to about 30 mg/kg body weight, or more. In specific embodiments, the dosage may range from 1 µg/kg to about 20 mg/kg, optionally from 10 µg/kg up to 10 mg/kg or from 100 µg/kg up to 5 mg/kg.

The term “effective dose” or “effective dosage” or “effective amount” is defined as an amount sufficient to achieve or at least partially achieve the desired effect. The term “effective dose” is meant to encompass an amount sufficient to cure or at least partially arrest the disease and its complications in a patient already suffering from the disease. Amounts or doses effective for this use will depend on the condition to be treated, the delivered antibody construct, the therapeutic context and objectives, the severity of the disease, prior therapy, the patient’s clinical history and response to the therapeutic agent, the route of administration, the size (body weight, body surface or organ size) and/or condition (the age and general health) of the patient, and the general state of the patient’s own immune system. The proper dose can be adjusted such that it can be administered to the patient once or over a series of administrations, and in order to obtain the optimal therapeutic effect.

Dosing for such purposes may be repeated as required, e.g. daily, semi-weekly, weekly, semi-monthly, monthly, or as required during relapses.

In an aspect, the invention relates to a method for selecting a specific anti-SIRPg compound, comprising or consisting of at least one of the following steps:
a. testing (e.g. according to a method described in the examples) the ability of compound to bind to SIRPg, and/or to the polypeptide of SEQ ID No: 1 described herein, and/or the epitope sequence consisting of or localized within the polypeptide of SEQ ID No: 1 as described herein;
b. testing (e.g. according to a method described in the examples) the ability of the compound to be an antagonist of the binding of SIRPg to CD47;
c. testing (e.g. according to a method described in the examples) the ability of the compound to bind to SIRPa;
d. testing (e.g. according to a method described in the examples) the ability of an antibody, an antigen-binding fragment or mimetic of such an antibody to decrease the binding of CD47 to SIRPa;
e. testing (e.g. according to a method described in the examples) the ability of the compound to bind to SIRPb;
and optionally comprising the following step:
selecting a compound which significantly inhibits the binding of human CD47 to human SIRPg, and which does not specifically bind to SIRPa and/or which does not inhibit or prevent the binding of CD47 to SIRPa, and/or which does not specifically bind to SIRPb. The selected anti-SIRPg compound may have any capability (e.g. at least one or several) illustrated for any specific anti-SIRPg compound already described herein, such as EC50 for SIRPg, SIRPa, SIRPb, and IC50 for the binding between SIRPg and CD47

In an aspect, the invention invention relates to a method for selecting a specific anti-SIRPg compound as disclosed here above, wherein the first step of the method is:

a′. testing (e.g. according to a method described in the examples) the ability of compound to bind to SIRPg, and/or to a polypeptide constituted of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, or the 29 consecutive amino acid residues of SEQ ID No: 1.

In an aspect, the invention invention relates to a method for selecting a specific anti-SIRPg compound as disclosed here above, wherein the first step of the method is:

a″. testing (e.g. according to a method described in the examples) the ability of compound to bind to SIRPg, and/or to a polypeptide consisting of at least 10 consecutive amino acid residues which share at least 80% identity with SEQ ID No: 1, with the proviso that the amino acid residues “E” and “N” localized respectively on positions 9 and 10 of SEQ ID No: 1, and/or the amino acid residue “P” localized on position 17 of SEQ ID No: 1, and/or the amino acid residues “M”, “A”, “L” and “G” localized respectively on positions 21 to 24 of SEQ ID No: 1, and/or the amino acid residues of SEQ ID No. 45, are present.

In an aspect, the invention invention relates to a method for selecting a specific anti-SIRPg compound as disclosed here above, wherein the first step of the method is:

a‴. testing (e.g. according to a method described in the examples) the ability of compound to bind to SIRPg, and/or to a polypeptide constituted of the amino acid residues of SEQ ID No. 45.

In an aspect, the invention also relates to an anti-SIRPg compound as defined above, for use in a diagnostic test, particularly in personalized medicine, more particularly in a companion diagnostic test.

In an embodiment, the invention relates to a method of diagnostic, particularly in personalized medicine, more particularly in a companion diagnostic test, using an anti-SIRPg compound as defined above.

In an embodiment, the invention also relates to a kit of diagnostic, particularly for personalized medicine, more particularly in a companion diagnostic kit, comprising an anti-SIRPg compound as defined above.

In an embodiment, the invention relates to an anti-SIRPg compound as defined above in the manufacture of a medicament for a diagnostic test, particularly in personalized medicine, more particularly in a companion diagnostic test.

In an aspect, the invention also relates to the use of at least one anti-SIRPg compound of the invention, in particular with anti-human SIRPg antibody or antigen-binding fragment thereof that does not cross react with human SIRPa, as a means for determination of the expression and/or level of expression of SIRPg in a biological sample previously obtained from a subject.

The invention also relates to an in vitro or ex vivo method to determine a SIRPg positive cells in a biological sample previously obtained from a subject, comprising:

i) determining in vitro the expression and/or the level of expression of SIRPg, in a biological sample of said subject using the anti- SIRPg compound of the invention in particular with anti-human SIRPg antibody or antigen-binding fragment thereof that does not cross react with human SIRPa, and in particular which does not cross react with human SIRPb.

The invention also relates to the use of at least one anti- SIRPg compound of the invention, in particular with anti-human SIRPg antibody or antigen-binding fragment thereof that does not cross react with human SIRPa, in particular which does not cross react with human SIRPa and human SIRPb, in a method wherein SIRPg is used as a biomarker that is predictive for the response to a treatment in a subject.

The invention also relates to an in vitro method of predicting the response of a subject to a treatment, in particular with a specific anti-SIRPg compound of the invention, in particular with anti-human SIRPg antibody or antigen-binding fragment thereof of the invention which does not cross react with human SIRPa, more particularly which does not cross react with human SIRPa and human SIRPb, comprising:
determining the expression level of SIRPg in a sample previously obtained from a subject, in particular a human subject,, with a a specific anti-SIRPg compound of the invention, and
comparing the expression level of SIRPg to a value representative of an expression level of SIRPg in a non-responding subject population,
wherein a higher expression level of SIRPg in the sample of the subject is indicative for a subject who will respond to the treatment.

The invention also concerns a method for treating or preventing a disease or a disorder in which T cells have a deleterious effect in a human subject, the method comprising the antagonism, in particular the prevention and/or the inhibition, of the binding of human CD47 to human SIRPg by administrating to the subject a specific anti-SIRPg compound of the invention. In a more particular embodiment, the invention concerns a method for treating or preventing a disease or a disorder in which T cells proliferation and/or activation and/or migration and/or tissues infiltration has(have) a deleterious effect in a human subject, the method comprising the antagonism, in particular the prevention and/or the inhibition, of the binding of human CD47 to human SIRPg by administrating to the subject a specific anti-SIRPg compound of the invention. In a more particular embodiment, the disease or disorder in which T cells have a deleterious effect is selected from the group consisting of:
an auto-immune disease, in particular rheumatoid arthritis, type I diabetes, lupus, psoriasis,
a chronic inflammatory disease, in particular Inflammatory bowel diseases including Crohn disease and Ulcerative colitis,
a chronic neuroinflammatory disease, in particular multiple sclerosis, encephalomyelitis,
an immune-metabolic disease, in particular type II diabetes,
a cardiovascular disease caused by a systemic inflammation, in particular atherosclerosis,
a transplant dysfunction or rejection, in particular Graft-versus-Host disease, and
a lymphoproliferative disease, in particular T cell lymphoma or post-transplant lymphoproliferative disease.

In a particular embodiment of the method, the administration of an anti-SIRPg compound of the invention decreases or inhibits the proliferation of T cells over 20% as compared with a negative control, more particularly more than 50%, and most preferably more than 70%.

In a particular embodiment of the method, the disease or disorder in which T cell proliferation and/or activation and/or migration and/or tissues infiltration has(have) a deleterious effect is selected from the group consisting of:
an auto-immune disease, in particular rheumatoid arthritis, type I diabetes, lupus, psoriasis,
a chronic neuroinflammatory disease, in particular multiple sclerosis, encephalomyelitis.

In a particular embodiment of the method, the disease or disorder in which T cell proliferation and/or activation and/or migration and/or tissues infiltration has(have) a deleterious effect is a transplant dysfunction, in particular graft-versus-host disease, in particular T cell lymphoma or post-transplant lymphoproliferative disease.

The invention also concerns a method for treating or preventing a disease or a disorder in which T cell, more particularly in which T cell proliferation and/or activation and/or migration and/or tissues infiltration, has(have) a deleterious effect in a human subject, the method comprising the antagonism, in particular the prevention and/or the inhibition, of the binding of human CD47 to human SIRPg by administrating to the subject a specific anti-SIRPg compound of the invention, wherein the disease or disorder in which T cell proliferation has a deleterious effect is selected from the group consisting of:
an auto-immune disease, in particular rheumatoid arthritis, type I diabetes, lupus, psoriasis,
a chronic inflammatory disease, in particular Inflammatory bowel diseases including Crohn disease and Ulcerative colitis,
a chronic neuroinflammatory disease, in particular multiple sclerosis, encephalomyelitis,
an immune-metabolic disease, in particular type II diabetes,
a cardiovascular disease caused by a systemic inflammation, in particular atherosclerosis,
a transplant dysfunction or rejection, in particular Graft-versus-host disease, and
a lymphoproliferative disease, in particular T cell lymphoma or post-transplant lymphoproliferative disease.
wherein the specific anti-SIRPg compound of the invention is administered in combination with at least one second therapeutic agent selected from the group consisting of immunotherapeutic agents, immunosuppressive agents, antibiotics and probiotics, said administration in combination being either simultaneous, separate or sequential.

In a particular embodiment, the immunosuppressive agent is selected from the group consisting of Cyclosporine A, tacrolimus, mycophenolate mofetil, rapamycine, steroids, anti-TNF agents, anti-IL-23 agents.

In a particular embodiment of the method, the method comprises an in vitro or ex vivo prediction of the response to a treatment in a subject, said prediction comprising measuring the expression level of SIRPg in a sample from a subject receiving the treatment or likely to receive the treatment, said expression level being determined with an anti-SIRPg compound of the invention, said prediction further comprising the comparison of the level of expression of SIRPg to a value representative of an expression level of SIRPg in a non-responding subject population, wherein a higher expression level of SIRPg in the sample of the subject is indicative for the subject who will respond to the treatment.

The invention also relates to the use of a specific anti-SIRPg compound selected from the group consisting of an antibody, an antigen-binding fragment thereof and an antigen binding antibody mimetic, which specifically binds to an epitope of human SIRPg consisting of or localized within the polypeptide of sequence SEQ ID No: 1; which is an antagonist of the binding of human SIRPg to human CD47 in the manufacture of a medicament for the prevention and/or treatment of a disease or a disorder, in particular a human disease or a human disorder, in which T cells have a deleterious effect.

The invention also relates to the use of a specific anti-SIRPg compound selected from the group consisting of an antibody, an antigen-binding fragment thereof and an antigen binding antibody mimetic, which specifically binds to an epitope of human SIRPg consisting of or localized within the polypeptide of sequence SEQ ID No: 1; which is an antagonist of the binding of human SIRPg to human CD47 in the manufacture of a medicament for the prevention and/or treatment of a disease or a disorder, in particular a human disease or a human disorder, in which the proliferation and/or the activation and/or the migration of T cells and/or the tissues infiltration by T cells has (have) a deleterious effect.

The invention also relates to the use of a specific anti-SIRPg compound selected from the group consisting of an antibody, an antigen-binding fragment thereof and an antigen binding antibody mimetic, which specifically binds to an epitope of human SIRPg consisting of or localized within the polypeptide of sequence SEQ ID No: 1; which is an antagonist of the binding of human SIRPg to human CD47 in the manufacture of a medicament for the prevention and/or treatment of a disease or a disorder, in particular a human disease or a human disorder, selected from the group consisting of:
an auto-immune disease, in particular rheumatoid arthritis, type I diabetes, lupus, psoriasis,
a chronic inflammatory disease, in particular Inflammatory bowel diseases including Crohn disease and Ulcerative colitis,
a chronic neuroinflammatory disease, in particular multiple sclerosis, encephalomyelitis,
an immune-metabolic disease, in particular type II diabetes,
a cardiovascular disease caused by a systemic inflammation, in particular atherosclerosis,
a transplant dysfunction or rejection, in particular Graft-versus-host disease, and
a lymphoproliferative disease, in particular T cell lymphoma or post-transplant lymphoproliferative disease.

The invention also relates to the use of a polypeptide corresponding to any epitope sequence disclosed therein, for assessing if an anti-SIRPg compound is suitable for antagonizing the binding of human SIRPg to human CD47, and in particular which does not prevent or inhibit the binding of SIRPa to CD47, and/or in particular which does not bind to human SIRPa, and/or which does not bind to human SIRPb.

In a more particular embodiment, the polypeptide used for assessing if an anti-SIRPg compound is suitable for antagonizing the binding of human SIRPg to human CD47, and in particular which does not prevent or inhibit the binding of SIRPa to CD47, and/or in particular which does not specifically bind to human SIRPa, and/or which does not specifically bind to human SIRPb is the polypeptide of SEQ ID No: 1.

The invention also relates to a method for purifying a specific anti-SIRPg compound, which is suitable for antagonizing the binding of human SIRPg to human CD47, said method comprising:
Coating a surface with a polypeptide comprising or consisting of the amino acid of SEQ ID No: 1; or of SEQ ID No. 45, for example, the polypeptide may comprise an extra-histidine tail used for coating the polypeptide on a surface;
Incubating a compound with the surface coated with the polypeptide;
Possibly measuring the binding of the compound to the polypeptide;
Possibly testing the binding of the compound to the polypeptide in presence of CD47;
Retrieving the anti-SIRPg compound with the desired binding capability for the polypeptide.

The binding measures may be performed according to the methods described in the examples of the present invention. The desired binding capability may be the one defined for the specific anti-SIRPg compound of the invention here above (e.g. EC50 for the polypeptide similar to the EC50 for the binding to SIRPg). A further step of testing the capability of the retrieved compound to antagonize the binding ot SIRPg to CD47 may be performed, for example as illustrated in the examples of the invention.

The invention also relates to a method for selecting a specific anti-SIRPg compound which
binds specifically to human SIRPg, and antagonizes the binding of human SIRPg to human CD47;
does not prevent or inhibit the binding of human SIRPa to human CD47, in particular which does not specifically bind to human SIRPa; and
optionally which does not specifically bind to human SIRPb;
wherein the method comprises a step of testing the ability of the compound to bind to any epitope sequence as disclosed therein; more particularly a step of testing the ability of the compound to bind to the polypeptide of SEQ ID No: 1, or of SEQ ID No. 45, a compound being a specific anti-SIRPg compound when it specifically binds to said polypeptide.

The invention also relates to a specific anti-SIRPg compound which binds specifically to human SIRPg and which is an antagonist of the binding of human SIRPg to human CD47, wherein the anti-SIRPg compound cross-competes for binding to a polypeptide consisting of SEQ ID No:1, or of SEQ ID No. 45, with an antibody which comprises:
an antibody heavy chain variable domain comprising:
   VHCDR1 of SEQ ID No: 17 or SEQ ID No: 29;
   VHCDR2 of SEQ ID No: 34; SEQ ID No: 19 or SEQ ID No: 31;
   o VHCDR3 of SEQ ID No: 21; SEQ ID No: 25 or SEQ ID No: 33; and
an antibody light chain variable domain comprising:
   VLCDR1 of SEQ ID No: 9;
   VLCDR2 of SEQ ID No: 11;
   VLCDR3 of SEQ ID No: 13;
in particular the anti-SIRPg compound cross-competes for binding to a polypeptide consisting of SEQ ID No: 1, or of SEQ ID No. 45, with an antibody comprising:
a heavy chain variable domain comprising or consisting of the polypeptide of SEQ ID No: 15; SEQ ID No: 23 or SEQ ID No: 27; and
a light chain variable domain comprising or consisting the polypeptide of SEQ ID No: 7.

Also encompassed by the present invention is a specific anti-SIRPg compound which competes for binding to a polypeptide of SEQ ID No:1 with an antibody comprising:
an antibody heavy chain variable domain comprising:
   VHCDR1 of SEQ ID No: 17 or SEQ ID No: 29;
   VHCDR2 of SEQ ID No: 34; SEQ ID No: 19 or SEQ ID No: 31;
   VHCDR3 of SEQ ID No: 21; SEQ ID No: 25 or SEQ ID No: 33; and
an antibody light chain variable domain comprising:
   VLCDR1 of SEQ ID No: 9;
   VLCDR2 of SEQ ID No: 11;
   VLCDR3 of SEQ ID No: 13;
in particular the specific anti-SIRPg compound cross-competes for binding to a polypeptide consisting of SEQ ID No: 1 with an antibody comprising:
a heavy chain variable domain comprising or consisting of the polypeptide of SEQ ID No: 15; SEQ ID No: 23 or SEQ ID No: 27; and
a light chain variable domain comprising or consisting the polypeptide of SEQ ID No: 7.

Cross-competing antibodies (or compounds) and antibodies (or compounds) that recognize the same or an overlapping epitope localized (or present or comprised) within the polypeptide of SEQ ID No:1 can be identified using routine techniques such as an immunoassay, for example, by showing the ability of one antibody to block the binding of another antibody to a target antigen, e.g., a competitive binding assay. Competitive binding may be determined using an assay such as described in the examples of the present invention. Cross-competition is present if the tested anti-SIRPg compound reduces binding of the other antibody by at least by 50%, at least by 60%, specifically at least by 70% and more specifically at least by 80% and vice versa in comparison to the positive control which lacks one of said antibodies (or compounds).

The present invention also encompasses a method for selecting an anti-SIRPg compound, which binds specifically to human SIRPg and which is an antagonist of the binding of human SIRPg to human CD47, said method comprising:
Testing the cross-competition for binding to a polypeptide consisting of SEQ ID No:1, or of SEQ ID No. 45, between the anti-SIRPg compound to be selected and a specific anti-SIRPg antibody comprising:
   an antibody heavy chain variable domain comprising:
      VHCDR1 of SEQ ID No: 17 or SEQ ID No: 29;
      VHCDR2 of SEQ ID No: 34; SEQ ID No: 19 or SEQ ID No: 31;
      VHCDR3 of SEQ ID No: 21; SEQ ID No: 25 or SEQ ID No: 33; and
   an antibody light chain variable domain comprising:
      VLCDR1 of SEQ ID No: 9;
      VLCDR2 of SEQ ID No: 11;
      VLCDR3 of SEQ ID No: 13;
Selecting an anti-SIRPg compound which shown at least similar binding capability to the polypeptide as compared to the specific anti-SIRPg antibody used in the cross-competition assay (e.g. similar EC50 for SIRPa and SIRPg as the values defined for the recited antibodies), and which shown at least similar inhibition capability for the interaction between SIRPg and CD47 as compared to the specific anti-SIRPg antibody used in the cross-competition assay (e.g. similar IC 50 as the values defined for the recited antibodies).

The invention also relates to a method for producing a specific anti-SIRPg antibody which is suitable for antagonizing the binding of human SIRPg to human CD47, said method comprising immunizing a non-human mammal with an antigen comprising an epitope sequence within the polypeptide consisting of SEQ ID No: 1, in particular with an antigen consisting of or localized within SEQ ID No: 1. Such a method may further comprise obtaining a hybridoma after immunizing an animal, and, where necessary, boosting said animal with the same immunogen, recovering spleen or lymph node cells from the animal responding to immunization and fusing said cells with myeloma cells to isolate monoclonal antibodies and / or expressing polynucleotides coding for such antibodies such as polynucleotides disclosed herein with their nucleotide sequence in the recombinant form in cells in conditions enabling the recovery of antibodies, and/or recovering antibodies specifically recognizing an epitope as defined herein, or the polypeptide of SEQ ID No: 1, in particular antibodies having the desired binding affinity for SIRPg, SIRPa, in particular for SIRPg, SIRPa and SIRPb, and which has the desired antagonist capability for the binding of SIRPg to CD47, in particular as compared with any antibody disclosed herein.

The invention also relates to a method for producing a specific anti-SIRPg antibody which is suitable for antagonizing the binding of human SIRPg to human CD47, wherein the method comprises the immunization of a non-human mammal with one, or several, antigens selected from the group consisting of:
a consisting of or localized within the polypeptide of SEQ ID No: 1 as described herein;
a polypeptide constituted of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, or the 29 consecutive amino acid residues of SEQ ID No: 1.
a polypeptide consisting of at least 10 consecutive amino acid residues which share at least 80% identity with SEQ ID No: 1, with the proviso that the amino acid residues “E” and “N” localized respectively on positions 9 and 10 of SEQ ID No: 1, and/or the amino acid residue “P” localized on position 17 of SEQ ID No: 1, and/or the amino acid residues “M”, “A”, “L” and “G” localized respectively on positions 21 to 24 of SEQ ID No: 1, and/or the amino acid residues of SEQ ID No. 45, are present
a polypeptide constituted of the amino acid residues of SEQ ID No. 45.

The following Figures and Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### FIGURE LEGENDS

FIG. 1. Binding on peptide of SEQ ID No: 1 in ELISA assay peptide SIRPg-His coating and anti-human peroxidase detection. Binding analysis of antibodies A1, A5 and A8, and prior art antibody Kwar23.

A. ELISA of Kwar23 (■), A1 (▲), A5 (x) and A8 (□) on specific peptide SIRPg-His immobilized at 50 µg/ml. B. ELISA of LSB2.20 (x) on specific peptide SIRPg-His immobilized at 50 pg/ml. Revelation was performed with a donkey anti-human antibody and revealed by colorimetry at 450 nm using TMB substrate. C. EC50 of the antibodies. EC50 is the concentration of the indicated antibody to reach 50% of the signal in this assay.

FIG. 2. Binding on SIRPg, SIRPa and SIRPb ELISA assay (protein SIRPa/b/g-His coated and anti-human peroxidase detection). Binding analysis of antibodies A1, A5 and A8, and prior art antibody Kwar23.

ELISA of Kwar23 (■), A1 (▲), A5 (x) and A8 (◊) on protein SIRPg-His at 1 µg/ml immobilized on FIG. A, on protein SIRPa-His at 1 µg/ml immobilized on FIG. B, and on protein SIRPb-His at 1 µg/ml immobilized on FIG. C. Revelation was performed with a donkey anti-human antibody and revealed by colorimetry at 450 nm using TMB substrate.

FIG. 3. Binding on SIRPg, SIRPa, SIRPb and Peptide of SEQ ID No. 1 of prior art antibody OX119.

ELISA of OX119 at 1 µg/ml on immobilized peptide of SEQ ID No. 1, hSIRPa-His, hSIRPb-his and hSIRPg-His at 0.5 µg to 1 µg. Revelation was performed with a secondary antibody and revealed by colorimetry at 540-630 nm.

FIG. 4. Anti-SIRPg antagonist activity by competition ELISA assay with CD47. Binding analysis of (A) anti-SIRPg antibodies A1, A5 and A8, and antibodies of the prior art Kwar23, LSB2.20 and (B) OX119.

ELISA of Kwar23 (■), A1 (▲), A5 (x), A8 (◊) and commercial LSB2-20 (∗) and OX119 (•) at different concentrations incubated with constant concentration of biotinylated CD47-Fc (6 µg/ml). Revelation was performed with streptavidin peroxidase to detect CD47 molecule and revealed by colorimetry at 450 nm or 450-630 nm using TMB substrate. IC50 is the concentration of the indicated antibody required for inhibiting 50% of the signal in this assay.

### EXAMPLES

### Material and Methods

### Antibodies Generation

Anti-SIRPg antibodies were obtained according to the Phage Display technology. A semi-synthetic scFv library constructed similar to the library described in Nissim et al., (Nissim A, Hoogenboom HR, Tomlinson IM, Flynn G, Midgley C, Lane D, Winter G. EMBO J. 1994 Feb 1;13(3):692-8). The antibodies generated according to the phage display technology were subsequently screened according to their binding affinity to the peptide of SEQ No: 1.

### Preparation of Antibody

For construction of heavy chain of anti-SIRPgamma Ab, variable domain VH from A1, A5, or A8 sequence were synthetized and cloned by EcoRV in pFuseCHlg-hG4m expression plasmid containing Fc of human lgG4 mutated (S228P) to stabilize hinge region (pFuseCHlg-hG4m vector from Invivogen, Toulouse). For construction of light chain of anti-SIRPgamma Ab, the identical variable domain VL from A1, A5, or A8 sequence was synthetized and cloned by Accl/Nhel in pFuse2CLlg-hk expression plasmid containing human CLkappa (pFuse2CLlg-hk from Invivogen, Toulouse). In HEK 293 Freestyle cells, we have co-transfected, by lipofectamine method, plasmids containing VHA1-hFcG4 or VHA5-hFcG4 or VHA8-hFcG4 with plasmid containing VL-CLkappa. After 48-72 h incubation, supernatant was recovered and purified by affinity on Protein A chromatography (HiTrap, GeHealthcare) with citric acid 0.1 M pH 3 elution buffer. Purified antibody was dialyzed in PBS and concentrated. They were quantified by UV (A280nm) and tested in activity assay against SIRPg like antigen.

### ELISA Binding Anti-SIRP_{g} (Peptide)

For activity ELISA assay, peptide human SIRPgamma (VKFRKGSPENVEFKSGPGTEMALGAKPSA-6His, synthetised by Synpeptide, Shanghai, China) was immobilized on plastic at 100 µg/ml in carbonate buffer (pH 9.2) and purified antibody were added to measure binding. After incubation and washing, peroxidase-labeled donkey anti-human IgG (Jackson Immunoresearch; USA; reference 709-035-149) was added and revealed by conventional methods.

### ELISA Binding Anti-SiRPg / SIRPa (Recombinant Protein)

For activity ELISA assay, recombinant hSIRPg (Sino Biologicals, Beijing, China; reference 11828-H08H) or hSIRPa (Sino Biologicals, Beijing, China; reference 11612-H08H) was immobilized on plastic at 1 µg/ml (hSIRPg) or 0.5 µg/ml (hSIRPa) in carbonate buffer (pH9.2) and purified antibody were added to measure binding. After incubation and washing, peroxidase-labeled donkey anti-human IgG (Jackson Immunoresearch; USA; reference 709-035-149) was added and revealed by conventional methods.

### ELISA Antagonist: Competition Between CD47 and Anti-SiRPg Antibody

For competitive ELISA assay, recombinant hSIRPg (Sino Biologicals, Beijing, China; reference 11828-H08H) was immobilized on plastic at 1 µg/ml in carbonate buffer (pH9.2). Purified antibody (at different concentrations) were mixed with 6 µg/ml final (fix concentration) of biotinylated Human CD47Fc (AcroBiosystems interchim; France; reference: #CD7-H82F6) to measure competitive binding for 2 h at 37° C. After incubation and washing, peroxidase-labeled streptavidin (Vector laboratoring; USA; reference SA-5004) was added to detect Biotin-CD47Fc binding and revealed by conventional methods.

### Affinity for SiRPg by Blitz Method

This method was performed with a Blitz (Forté Bio; USA; reference C22-2 No 61010-1). Recombinant hSIRPg-His (Sino Biologicals, Beijing, China; reference 11828-H08H) was immobilized at 10 µg/ml by histidine tail into a Ni-NTA biosensor (Forté Bio; USA; reference 18-0029) for 30 seconds. Then, anti-SIRP antibodies were associated at 10 or 20 µg/mL for 120 seconds. The dissociation of anti-SIRPa antibody was made in kinetics buffer for 120 seconds. Analysis data was made with the Blitz pro 1.2 software, which calculated association constant (ka) and dissociation constant (kd) and determined the affinity constant KD (ka/kd).

### Manufacture and Selection of Anti-SiRPg Antibodies

Following the manufacture of the antibodies as described here above, a polypeptide with the amino acid sequence of SEQ ID No. 1 has been used for testing the abilities of different anti-SIRPg compounds, including prior art antibodies, and home-made antibodies. The home-made antibodies correspond to the structure detailed in the following tables and detailed explanations here under.

TABLE 1

Sequences of the specific CDRs of anti-SIRPq antibodies (according to the Kabat system)

| CDRs | Sequences | SEQ ID No. |
|---|---|---|
| HCDR1-A1/A5 | DDYGMS | 17 |
| HCDR1-A8 | SSYAMS | 29 |
| HCDR2-A1/A5 | GINWNGGSTGYADSVKG | 19 |
| HCDR2-A8 | AISGSGGSTYYADSVKG | 31 |
| HCDR2-cons | X₁IX₂X₃X₄GGSTX₅YADSVKG, wherein X₁ is A or G, X₂ is S or N, X₃ is G or W, X₄ is S or N, X₅ is G or Y | 34 |
| HCDR3-A1 | WIAKV | 21 |
| HCDR3-A5 | QMVKL | 25 |
| HCDR3-A8 | RQYLGVL | 33 |
| LCDR1-comm | QGDSLRSYYAS | 9 |
| LCDR2-comm | GKNNRPS | 11 |
| LCDR3-comm | CNSRDSSGNHVV | 13 |

TABLE 2

Sequences of chain variable domains of anti-SiRPg antibodies

| Variable domain | Sequences | SEQ ID No. |
|---|---|---|
| H-A1 | EVQLVESGGGVVRPGGSLRLSCAASGFTFDDYGMSWVRQ APGKGLEWVSGINWNGGSTGYADSVKGRFTISRDNAKNSL YLQMNSLRAEDTAVYYCARWIAKVWGQGTLVTVSR | 15 |
| H-A5 | EVQLVESGGGVVRPGGSLRLSCAASGFTFDDYGMSWVRQ APGKGLEWVSGINWNGGSTGYADSVKGRFTISRDNAKNSL YLQMNSLRAEDTAVYYCARQMVKLWGQGTLVTVSR | 23 |
| H-A8 | EVQLVESGGGLVQPGGSLRLSCAASGFTFSSYAMSWVRQ APGKGLEWVSAISGSGGSTYYADSVKGRFTISRDNSKNTL YLQMNSLRAEDTAVYYCARRQYLGVLWGQGTLVTVSR | 27 |
| L-comm | SSELTQDPAVSVALGQTVRITCQGDSLRSYYASWYQQKPG QAPVLVIYGKNNRPSGIPDRFSGSSSGNTASLTITGAQAED EADYYCNSRDSSGNHVVFGGGTKLTVL | 7 |

TABLE 3

Sequences of full chains of antibodies

| Full chains | Sequences | SEQ ID No. |
|---|---|---|
| H-full-A1 | EVQLVESGGGVVRPGGSLRLSCAASGFTFDDYGMSWVRQ APGKGLEWVSGINWNGGSTGYADSVKGRFTISRDNAKNSL YLQMNSLRAEDTAVYYCARWIAKVWGQGTLVTVSRASTKG PSVFPLAPCSRSTSESTAALGCLVKDYFPEPVTVSWNSGA LTSGVHTFPAVLQSSGLYSLSSVVTVPSSSLGTKTYTCNVD HKPSNTKVDKRVESKYGPPCPPCPAPEFLGGPSVFLFPPK PKDTLMISRTPEVTCVVVDVSQEDPEVQFNWYVDGVEVHN AKTKPREEQFNSTYRVVSVLTVLHQDWLNGKEYKCKVSNK GLPSSIEKTISKAKGQPREPQVYTLPPSQEEMTKNQVSLTC LVKGFYPSDIAVEWESNGQPENNYKTTPPVLDSDGSFFLY SRLTVDKSRWQEGNVFSCSVMHEALHNHYTQKSLSLSPG K | 15 + 3 |
| H-full-A5 | EVQLVESGGGVVRPGGSLRLSCAASGFTFDDYGMSWVRQ APGKGLEWVSGINWNGGSTGYADSVKGRFTISRDNAKNSL YLQMNSLRAEDTAVYYCARQMVKLWGQGTLVTVSRASTK GPSVFPLAPCSRSTSESTAALGCLVKDYFPEPVTVSWNSG ALTSGVHTFPAVLQSSGLYSLSSVVTVPSSSLGTKTYTCNV DHKPSNTKVDKRVESKYGPPCPPCPAPEFLGGPSVFLFPP KPKDTLMISRTPEVTCVVVDVSQEDPEVQFNWYVDGVEVH NAKTKPREEQFNSTYRVVSVLTVLHQDWLNGKEYKCKVSN KGLPSSIEKTISKAKGQPREPQVYTLPPSQEEMTKNQVSLT CLVKGFYPSDIAVEWESNGQPENNYKTTPPVLDSDGSFFL YSRLTVDKSRWQEGNVFSCSVMHEALHNHYTQKSLSLSP GK | 23 + 3 |
| H-full-A8 | EVQLVESGGGLVQPGGSLRLSCAASGFTFSSYAMSWVRQ APGKGLEWVSAISGSGGSTYYADSVKGRFTISRDNSKNTL YLQMNSLRAEDTAVYYCARRQYLGVLWGQGTLVTVSRAS TKGPSVFPLAPCSRSTSESTAALGCLVKDYFPEPVTVSWN SGALTSGVHTFPAVLQSSGLYSLSSVVTVPSSSLGTKTYTC NVDHKPSNTKVDKRVESKYGPPCPPCPAPEFLGGPSVFLF PPKPKDTLMISRTPEVTCVVVDVSQEDPEVQFNWYVDGVE VHNAKTKPREEQFNSTYRVVSVLTVLHQDWLNGKEYKCKV SNKGLPSSIEKTISKAKGQPREPQVYTLPPSQEEMTKNQVS LTCLVKGFYPSDIAVEWESNGQPENNYKTTPPVLDSDGSF FLYSRLTVDKSRWQEGNVFSCSVMHEALHNHYTQKSLSLS PGK | 27 + 3 |
| L-full-comm | SSELTQDPAVSVALGQTVRITCQGDSLRSYYASWYQQKPG QAPVLVIYGKNNRPSGIPDRFSGSSSGNTASLTITGAQAED EADYYCNSRDSSGNHVVFGGGTKLTVLRTVAAPSVFIFPPS DEQLKSGTASVVCLLNNFYPREAKVQWKVDNALQSGNSQ ESVTEQDSKDSTYSLSSTLTLSKADYEKHKVYACEVTHQGL SSPVTKSFNRGEC | 7+5 |

TABLE 4

Sequences of the specific CDRs of anti-SIRPq antibodies (according to the IMGT system)

| CDRs | Sequences | SEQ ID No. |
|---|---|---|
| HCDR1-A1/A5 | GFTFDDYG | 35 |
| HCDR2-A1/A5 | INWNGGST | 36 |
| HCDR3-A1 | ARWIAKV | 37 |
| HCDR1-A8 | GFTFSSYA | 38 |
| HCDR2-A8 | ISGSGGSTY | 39 |
| HCDR3-A8 | ARRQYLGVL | 40 |
| HCDR3-A5 | ARQMVKL | 44 |
| LCDR1-comm | SLRSYY | 41 |
| LCDR2-comm | GKN | 42 |
| LCDR3-comm | NSRDSSGNHVV | 43 |

The home-made antibodies which specifically binds to human SIRPg comprise the following structure:
an antibody heavy chain variable domain comprising:
   VHCDR1 of SEQ ID No: 17 or SEQ ID No: 29;
   VHCDR2 of SEQ ID No: 34; SEQ ID No: 19 or SEQ ID No: 31;
   VHCDR3 of SEQ ID No: 21; SEQ ID No: 25 or SEQ ID No: 33; and
an antibody light chain variable domain comprising:
   VLCDR1 of SEQ ID No: 9;
   VLCDR2 of SEQ ID No: 11;
   VLCDR3 of SEQ ID No: 13; and
These antibodies are antagonists of the binding of human SIRPg to human CD47.

More particularly, the home-made antibodies have the following structure:
an antibody heavy chain variable domain comprising:
   VHCDR1 of SEQ ID No: 17; VHCDR2 of SEQ ID No: 19 and VHCDR3 of SEQ ID No: 21; corresponding to the VHCDRs of the antibody A1 used in the examples of the present invention; or
   VHCDR1 of SEQ ID No: 17; VHCDR2 of SEQ ID No: 19 and VHCDR3 of SEQ ID No: 25; corresponding to the VHCDRs of the antibody A5 used in the examples of the present invention; or
   VHCDR1 of SEQ ID No: 29; VHCDR2 of SEQ ID No: 31 and VHCDR3 of SEQ ID No: 33; corresponding to the VHCDRs of the antibody A8 used in the examples of the present invention, and
   an antibody light chain variable domain comprising VLCDR1 of SEQ ID No: 9; VLCDR2 of SEQ ID No: 11 and VLCDR3 of SEQ ID No: 13; corresponding to the VLCDRs of antibodies A1, A5 and A8 used in the examples of the present invention.
More particularly, the home-made antibodies have the following structure:
A heavy chain variable domain of SEQ ID No: 15; corresponding to the heavy chain variable domain of antibody A1; or
A heavy chain variable domain of SEQ ID No: 23; corresponding to the heavy chain variable domain of antibody A5; or
A heavy chain variable domain of SEQ ID No: 27; corresponding to the heavy chain variable domain of antibody A8; and
A light chain variable domain of SEQ ID No: 7, corresponding to the light chain variable domain of antibodies A1, A5 and A8.
More particularly, the home-made antibodies have the following structure:
A heavy chain comprising or consisting of the combination of the Fc of human IgG4 of SEQ ID No: 3 with any one of the heavy chain variable domain of SEQ ID No: 15, SEQ ID No: 23 and SEQ ID No: 27; and
A light chain comprising or consisting of the combination of human CLkappa of SEQ ID No: 5 with the light chain variable domain of SEQ ID No: 7.
Such combinations correspond respectively to the antibodies A1, A5 and A8 used in the examples of the present invention.

Nucleic acid molecule(s) encoding home-made antibodies have been used, and comprise(s) the following structure(s);
A first acid molecule encoding a variable heavy chain domain and selected from:
A polynucleotide encoding a variable heavy chain domain and comprising the nucleotide residues of sequences SEQ ID No: 16, SEQ ID No: 18 and SEQ ID No: 20, encoding respectively the CDRs of SEQ ID No: 17; SEQ ID No: 19 and SEQ ID No: 21;
A polynucleotide encoding a variable heavy chain domain and comprising the nucleotide residues of sequences SEQ ID No: 16, SEQ ID No: 30 and SEQ ID No: 20, encoding respectively the CDRs of SEQ ID No: 17; SEQ ID No: 31 and SEQ ID No: 21;
A polynucleotide encoding a variable heavy chain domain and comprising the nucleotide residues of sequences SEQ ID No: 16, SEQ ID No: 18 and SEQ ID No: 24, encoding respectively the CDRs of SEQ ID No: 17; SEQ ID No: 19 and SEQ ID No: 25;
A polynucleotide encoding a variable heavy chain domain and comprising the nucleotide residues of sequences SEQ ID No: 16, SEQ ID No: 30 and SEQ ID No: 24, encoding respectively the CDRs of SEQ ID No: 17; SEQ ID No: 31 and SEQ ID No: 25;
A polynucleotide encoding a variable heavy chain domain and comprising the nucleotide residues of sequences SEQ ID No: 16, SEQ ID No: 18 and SEQ ID No: 32, encoding respectively the CDRs of SEQ ID No: 17; SEQ ID No: 19 and SEQ ID No: 33;
A polynucleotide encoding a variable heavy chain domain and comprising the nucleotide residues of sequences SEQ ID No: 16, SEQ ID No: 30 and SEQ ID No: 32, encoding respectively the CDRs of SEQ ID No: 17; SEQ ID No: 31 and SEQ ID No: 33;
A polynucleotide encoding a variable heavy chain domain and comprising the nucleotide residues of sequences SEQ ID No: 28, SEQ ID No: 18 and SEQ ID No: 20, encoding respectively the CDRs of SEQ ID No: 29; SEQ ID No: 19 and SEQ ID No: 21;
A polynucleotide encoding a variable heavy chain domain and comprising the nucleotide residues of sequences SEQ ID No: 28, SEQ ID No: 30 and SEQ ID No: 20, encoding respectively the CDRs of SEQ ID No: 29; SEQ ID No: 31 and SEQ ID No: 21;
A polynucleotide encoding a variable heavy chain domain and comprising the nucleotide residues of sequences SEQ ID No: 28, SEQ ID No: 18 and SEQ ID No: 24, encoding respectively the CDRs of SEQ ID No: 29; SEQ ID No: 19 and SEQ ID No: 25;
A polynucleotide encoding a variable heavy chain domain and comprising the nucleotide residues of sequences SEQ ID No: 28, SEQ ID No: 30 and SEQ ID No: 24, encoding respectively the CDRs of SEQ ID No: 29; SEQ ID No: 31 and SEQ ID No: 25;
A polynucleotide encoding a variable heavy chain domain and comprising the nucleotide residues of sequences SEQ ID No: 28, SEQ ID No: 18 and SEQ ID No: 32, encoding respectively the CDRs of SEQ ID No: 29; SEQ ID No: 19 and SEQ ID No: 32; and
A polynucleotide encoding a variable heavy chain domain and comprising the nucleotide residues of sequences SEQ ID No: 28, SEQ ID No: 30 and SEQ ID No: 32, encoding respectively the CDRs of SEQ ID No: 29; SEQ ID No: 31 and SEQ ID No: 33;
And a second acid molecule encoding a variable light chain and comprising the nucleotide residues of sequences SEQ ID No: 8, SEQ ID No: 10 and SEQ ID No: 12, encoding respectively the CDRs of SEQ ID No: 9; SEQ ID No: 11 and SEQ ID No: 13.

Nucleic acid molecules according to the invention may also be chosen from:
A polynucleotide encoding a variable heavy chain domain and comprising or consisting of the nucleotide residues of sequences SEQ ID No: 14, SEQ ID No: 22 or SEQ ID No: 26, encoding respectively the heavy chain domain of SEQ ID No: 15; SEQ ID No: 23 and SEQ ID No: 26; corresponding respectively to the heavy chain variable domains of antibodies A1, A5 and A8 disclosed in the examples of the present invention; and
A polynucleotide encoding a variable light chain domain and comprising or consisting of the nucleotide residues of sequences SEQ ID No: 6, encoding the light chain variable domain of SEQ ID No: 7, and corresponding to the light chain variable domains of antibodies A1, A5 and A8.

Nucleic acid molecules according to the invention may also be chosen from:
A polynucleotide encoding a full heavy chain and comprising or consisting of the nucleotide residues of sequences SEQ ID No: 2 and SEQ ID No: 14; or SEQ ID No: 2 and SEQ ID No: 22, or SEQ ID No: 2 and SEQ ID No: 26, encoding respectively the heavy chain of antibodies A1, A5 and A8; and
A polynucleotide encoding a full light chain and comprising or consisting of the nucleotide residues of sequences SEQ ID No: 4 and SEQ ID No: 6, encoding the light chain of antibodies A1, A5 and A8.

Such nucleic acid molecules may be inserted within an expression vector, like a plasmid for example, suitable for expression of the encoded sequence within a host cell.

### Example 1. Binding to Different Members of the SIRP Family

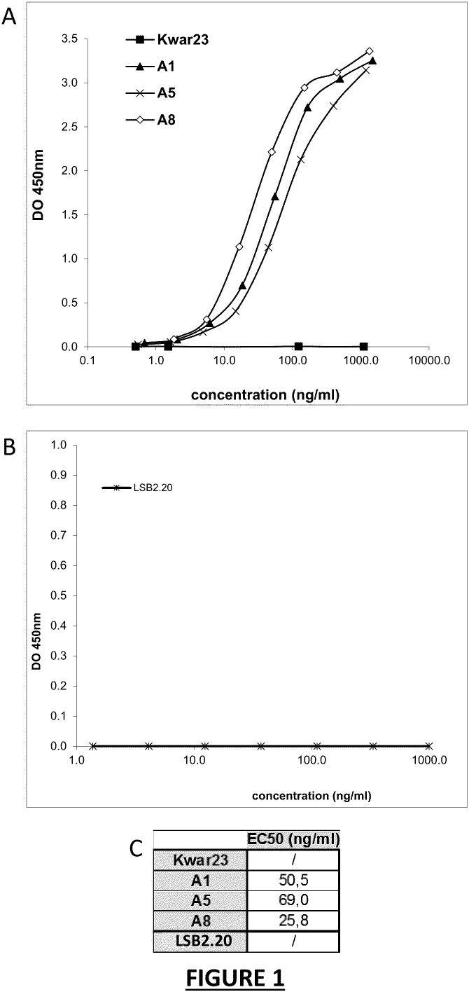

Results: As shown in FIG. 1, three antibodies A1, A5 and A8 have a good affinity binding for the peptide of SEQ ID No: 1, while the antibodies of the prior art Kwar23 and LSB2.20 do not bind to the peptide of SEQ ID No. 1.

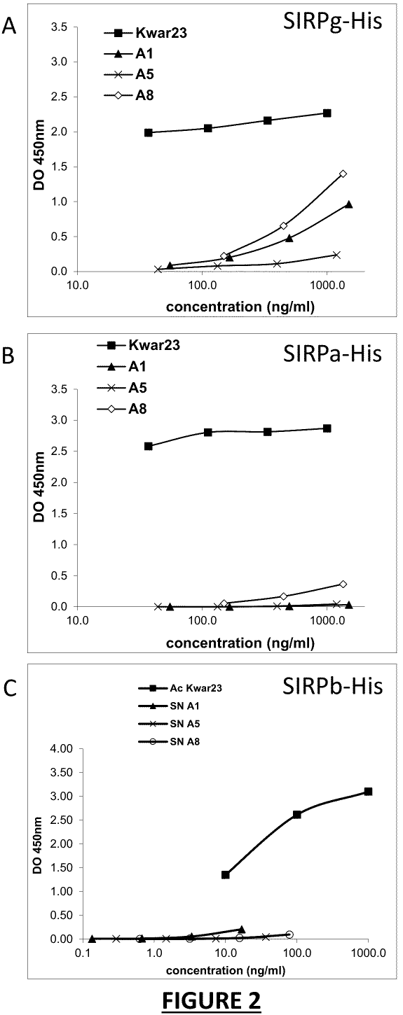

As shown in FIG. 2, three antibodies A1, A5 and A8 do bind to SIRPg (although with different binding behavior than antibody of prior art Kwar23), but do not recognize SIRPa or SIRPb, while Kwar23 is an anti-SIRPg antibody which also binds SIRPa and SIRPb. The three antibodies A1, A5 and A8 which bind to the amino acid peptide of SEQ ID No. 1 are selective for SIRPg whereas antibody of prior art Kwar23 is non-selective.

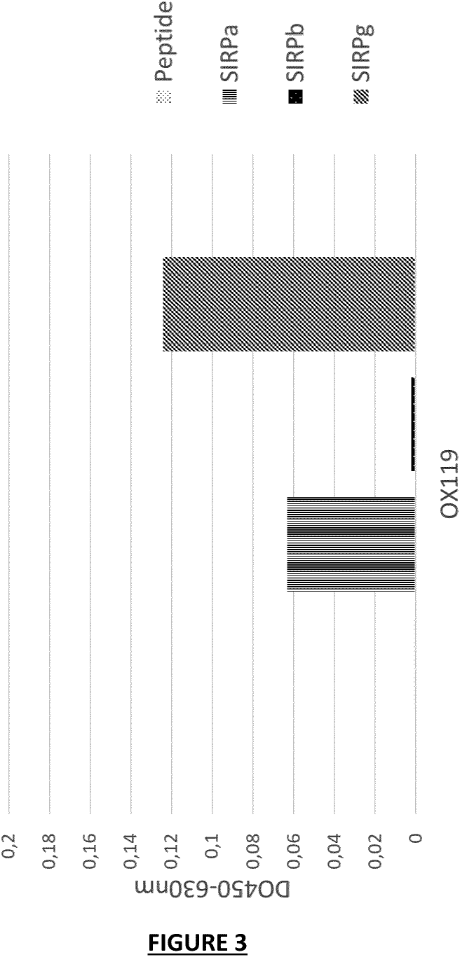

As shown in FIG. 3, the antibody of the prior art OX119 do bind to SIRPg, as expected, but also bind to SIRPa, while it is not able to recognize and bind the peptide correspond to the amino acid sequence of SEQ ID No. 1.

These results illustrates the unique ability of antibodies manufactured, and possibly selected, with an epitope sequence consisting of or localized within the amino acid sequence of SEQ ID No. 1 to selectively recognize and bind to a single member of the SIRP family, i.e. SIRPg, and to antagonize the binding of SIRPg to CD47, while anti-SIRPg antibodies of the prior art recognize at least two members of the SIRP family, i.e. SIRPa and/or SIRPb and/or are not able to antagonize the binding of SIRPg to CD47.

### Example 2. Ability to Inhibit the Binding of SIRPg to CD47

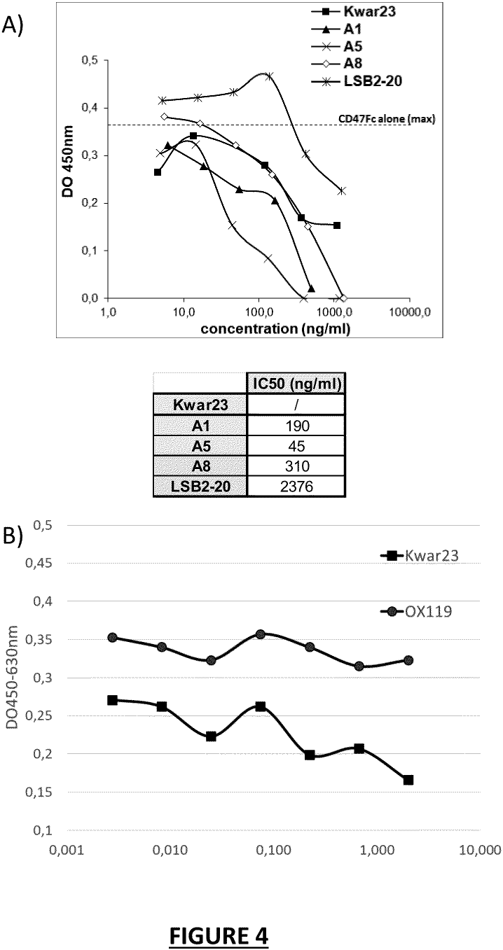

Results: As shown in FIG. 4, the three antibodies A1, A5 and A8are potent antagonists of the binding of SIRPg to CD47, while antibodies of the prior art Kwar23 and LSB2.20 and OX119 does not have the same antagonist effect. As illustrated on the FIG. 4a, and according to the IC50 values, a seven-time dose of LSB2.20 as compared with the antibodies A8 is required for reaching IC50 (and a 50-time dose as compared to antibody A5), while Kwar23 is not able to fully inhibit the binding of CD47 to SIRPg. As illustrated in FIG. 4b, OX119 does not have any impact on the binding between SIRPg ant CD47, since the binding of SIRPg to CD47 is not affected by any dose of OX119. Accordingly, anti-SIRPg compounds according to the invention are more potent for completely inhibiting the binding of SIRPg to CD47, and may elicit better efficiency in relation with the in vivo effect of anti-SIRPg antibodies (for example, inhibition of the proliferation of T Lymphocytes), and hence are suitable for enhancing the treatment or the prevention of the diseases, as discussed in the description of the invention.

1. A method for and/or treating a disease or a disorder in a human subjet comprising the step of: administering to the human subject a specific anti-Signal-Regulatory Protein gamma (SIRPg) compound, said compound being selected from the group consisting of an antibody, an antigen-binding fragment thereof and an antigen binding antibody mimetic, which specifically binds to an epitope of human SIRPg consisting of the polypeptide of sequence SEQ ID No: 45; which does not specifically bind to human Signal-Regulatory Protein alpha (SIRPa), and which is an antagonist of the binding of human SIRPg to human CD47,
wherein the disease or disorder to be treated and/or prevented is selected among the group consisting of:
an auto-immune disease
an inflammatory disease,
an immune-metabolic disease,
a transplant dysfunction or rejection and
a lymphoproliferative disease.

2. The method according to claim 1 wherein the proliferation and/or the activation and/or the migration of T cells and/or tissues infiltration by T cells has a deleterious effect.

3. (canceled)

4. The method according to claim 1, wherein the SIRPg compound does not prevent or inhibit the binding of human SIRPa to human CD47.

5. (canceled)

6. The method according to claim 1 where the SIRPg compound does not specifically bind to human Signal-Regulatory Protein beta (SIRPb).

7. The method according to claim 1 where the SIRPb compound dexreased or inhibits the proliferation of T cells as compared with a negative control.

8. The method according to claim 1 further comprising administering
at least one second therapeutic agent selected from the group consisting of immunotherapeutic agents, immunosuppressive agents, pro-apoptotic agents, antibiotics and probiotics, and mixtures thereof;
wherein the at least on second therapentic agent is administered simultaneous with, separate from or sequential to the at anti-SIRPg compound.

9. The method according to claim 8, wherein said immunosuppressive agent is selected from the group consisting of Cyclosporine A, tacrolimus, mycophenolate mofetil, rapamycine, steroids, anti-TNF agents, anti-IL-23 agents.

10. (canceled)

11. (canceled)

12. (canceled)

13. (canceled)

14. (canceled)

15. An in vitro method of predicting the response of a subject to a treatment comprising:
determining the expression level of SIRPg in a sample previously obtained from a subject with a specific anti-SIRPg compound specifically binding to a polypeptide consisting of SEQ ID No. 45; and
comparing the expression level of SIRPg to a value representative of an expression level of SIRPg in a non-responding subject population,
wherein a higher expression level of SIRPg in the sample of the subject is indicative for a subject who will respond to the treatment.

16. A method for selecting a specific anti-SIRPg compound, which:
binds specifically to human SIRPg, and which antagonizes inhibits the binding of human SIRPg to human CD47; and
does not prevent or inhibit the binding of human SIRPa to human CD47, and which does not specifically bind to human SIRPa; and
optionally which does not specicially bind to human SIRPb;
wherein the method comprises a step of testing the ability of the compound to bind to a polypeptide comprising or consisting of an epitope consisting of SEQ ID No: 45, a compound being a specific anti-SIRPg compound when it specifically binds to said epitope.

17. The compound for use according to claim 1, wherein in the presence of the compound at a concentration lower than 1000 ng/ml, the physical binding of human SIRPg to human CD47 is inhibited over 60% in an ELISA competition binding assay, as compared to a negative control.

18. The method of claim 1 wherein said disease or disorder is auto-immune disease selected from the group consisting of: rheumatoid arthritis, type I diabetes, lupus and psoriasis.

19. The method of claim 1 wherein said disease or disorder is graft-versus-host disease.

20. The method of claim 1 wherein said inflammatory disease is a chronic inflammatory disease or chronic neuroinflammatory disease.

21. The method of claim 1 wherein said disease or disorder is inflammatory bowel disease.

22. The method of claim 21 wherein said disease or disorder is Crohn’s disease or Ulcerative disease.

23. The method of claim 1, wherein said disease or disorder is a cardiovascular disease caused by a systemic inflammation.


SEQUENCE LISTING (45 sequences)

SEQ ID NO: 1 - protein, 29 aa, artificial sequence
  Synthetic sequence
VKFRKGSPENVEFKSGPGTEMALGAKPSA

SEQ ID NO: 2 - DNA, 984 bp, artificial sequence
  Synthetic sequence
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 3 - protein, 327 aa, artificial sequence
  Synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 4 - DNA, 324 bp, artificial sequence
  Synthetic sequence
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 107 aa, artificial sequence [sequence omitted]

SEQ ID NO: 6 - DNA, 324 bp, artificial sequence
  Synthetic sequence
tcatccgaactgactcaggatccagccgtgagcgtggctctgggacagaccgtgagaatcacatgccagggcgactccctgcgcagctactatgcctcttggtaccagcagaagccaggacaggctcccgtgctggtcatctacggcaagaacaataggccatctggcatccccgatcggttctccggatccagctctggaaacaccgcttccctgaccatcacaggagctcaggctgaggacgaggctgattactattgtaattcaagggatagtagcggtaatcatgtcgtgttcggcggcggcaccaaactgactgtcctg

SEQ ID NO: 7 - protein, 108 aa, artificial sequence
  Synthetic sequence
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 8 - DNA, 33 bp, artificial sequence
  Synthetic sequence
cagggcgactccctgcgcagctactatgcctct

SEQ ID NO: 9 - protein, 11 aa, artificial sequence
  Synthetic sequence
QGDSLRSYYAS

SEQ ID NO: 10 - DNA, 21 bp, artificial sequence
  Synthetic sequence
ggcaagaacaataggccatct

SEQ ID NO: 11 - protein, 7 aa, artificial sequence
  Synthetic sequence
GKNNRPS

SEQ ID NO: 12 - DNA, 36 bp, artificial sequence
  Synthetic sequence
tgtaattcaagggatagtagcggtaatcatgtcgtg

SEQ ID NO: 13 - protein, 12 aa, artificial sequence
  Synthetic sequence
CNSRDSSGNHVV

SEQ ID NO: 14 - DNA, 342 bp, artificial sequence
  Synthetic sequence
gaggtgcagctggtggagagcggaggaggagtggtgagaccaggaggatccctgaggctgagctgcgctgcttctggcttcacatttgacgattatggcatgtcttgggtgagacaggctccaggcaagggactggagtgggtgtccggcatcaactggaatggaggatccaccggatacgctgacagcgtgaagggcaggttcacaatctctcgggataacgccaagaattccctgtatctgcagatgaacagcctgagggctgaggacaccgccgtgtactattgtgctaggtggatcgctaaagtgtggggacagggcaccctggtgacagtgtctcgc

SEQ ID NO: 15 - protein, 114 aa, artificial sequence
  Synthetic sequence
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARWIAKVWGQGTLVTVSR

SEQ ID NO: 16 - DNA, 18 bp, artificial sequence
  Synthetic sequence
gacgattatggcatgtct

SEQ ID NO: 17 - protein, 6 aa, artificial sequence
  Synthetic sequence
DDYGMS

SEQ ID NO: 18 - DNA, 51 bp, artificial sequence
  Synthetic sequence
ggcatcaactggaatggaggatccaccggatacgctgacagcgtgaagggc

SEQ ID NO: 19 - protein, 17 aa, artificial sequence
  Synthetic sequence
GINWNGGSTGYADSVKG

SEQ ID NO: 20 - DNA, 15 bp, artificial sequence
  Synthetic sequence
tggatcgctaaagtg

SEQ ID NO: 21 - protein, 5 aa, artificial sequence
  Synthetic sequence
WIAKV

SEQ ID NO: 22 - DNA, 342 bp, artificial sequence
  Synthetic sequence
gaggtgcagctggtggagagcggaggaggagtggtgagaccaggaggatccctgaggctgagctgcgctgcttctggcttcacatttgacgattatggcatgtcttgggtgagacaggctccaggcaagggactggagtgggtgtccggcatcaactggaatggaggatccaccggatacgctgacagcgtgaagggcaggttcacaatctctcgggataacgccaagaattccctgtatctgcagatgaacagcctgagggctgaggacaccgccgtgtactattgtgcccggcagatggtgaagctgtggggccagggcaccctggtgacagtgtctcgc

SEQ ID NO: 23 - protein, 114 aa, artificial sequence
  Synthetic sequence
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARQMVKLWGQGTLVTVSR

SEQ ID NO: 24 - DNA, 15 bp, artificial sequence
  Synthetic sequence
cagatggtgaagctg

SEQ ID NO: 25 - protein, 5 aa, artificial sequence
  Synthetic sequence
QMVKL

SEQ ID NO: 26 - DNA, 348 bp, artificial sequence
  Synthetic sequence
gaggtgcagctggtggagtccggaggaggactggtgcagccaggaggaagcctgaggctgtcttgcgctgcttccggcttcaccttttccagctatgctatgtcttgggtgcggcaggctccaggcaagggactggagtgggtgtccgctatctctggctccggcggctccacatactatgccgacagcgtgaagggcagattcaccatcagccgcgataactctaagaatacactgtacctgcagatgaacagcctgagagctgaggacacagccgtgtactattgtgctaggcggcagtatctgggcgtgctgtggggacagggcaccctggtgacagtgtctcgc

SEQ ID NO: 27 - protein, 116 aa, artificial sequence
  Synthetic sequence
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARRQYLGVLWGQGTLVTVSR

SEQ ID NO: 28 - DNA, 18 bp, artificial sequence
  Synthetic sequence
tccagctatgctatgtct

SEQ ID NO: 29 - protein, 6 aa, artificial sequence
  Synthetic sequence
SSYAMS

SEQ ID NO: 30 - DNA, 51 bp, artificial sequence
  Synthetic sequence
gctatctctggctccggcggctccacatactatgccgacagcgtgaagggc

SEQ ID NO: 31 - protein, 17 aa, artificial sequence
  Synthetic sequence
AISGSGGSTYYADSVKG

SEQ ID NO: 32 - DNA, 21 bp, artificial sequence
  Synthetic sequence
cggcagtatctgggcgtgctg

SEQ ID NO: 33 - protein, 7 aa, artificial sequence
  Synthetic sequence
RQYLGVL

SEQ ID NO: 34 - protein, 17 aa, artificial sequence
  Synthetic sequence
  misc_feature (1)..(1): Xaa can be any naturally occurring amino acid
  misc_feature (3)..(5): Xaa can be any naturally occurring amino acid
  misc_feature (10)..(10): Xaa can be any naturally occurring amino acid
XIXXXGGSTXYADSVKG

SEQ ID NO: 35 - protein, 8 aa, Artificial Sequence
  synthetic sequence
GFTFDDYG

SEQ ID NO: 36 - protein, 8 aa, Artificial Sequence
  Synthetic sequence
INWNGGST

SEQ ID NO: 37 - protein, 7 aa, Artificial Sequence
  Synthetic sequence
ARWIAKV

SEQ ID NO: 38 - protein, 8 aa, Artificial Sequence
  Synthetic sequence
GFTFSSYA

SEQ ID NO: 39 - protein, 9 aa, Artificial Sequence
  Synthetic sequence
ISGSGGSTY

SEQ ID NO: 40 - protein, 9 aa, Artificial Sequence
  Synthetic sequence
ARRQYLGVL

SEQ ID NO: 41 - protein, 6 aa, Artificial Sequence
  Synthetic sequence
SLRSYY

SEQ ID NO: 42 - protein, 3 aa, Artificial Sequence
  Synthetic sequence
GKN

SEQ ID NO: 43 - protein, 11 aa, Artificial Sequence
  Synthetic sequence
NSRDSSGNHVV

SEQ ID NO: 44 - protein, 7 aa, Artificial Sequence
  Synthetic sequence
ARQMVKL

SEQ ID NO: 45 - protein, 7 aa, Artificial Sequence
  Synthetic sequence
KGSPENV